(19) United States
(12) Patent Application Publication
(10) Pub. No.: US 2026/0183004 A1
(43) Pub. Date: Jul. 2, 2026

(54) INTRAVASCULAR LITHOTRIPSY DEVICE

(71) Applicant: IV-X Medical, LLC, MENLO PARK, CA (US)

(72) Inventors: Ricardo David Roman, Chula Vista, CA (US); Alexander Joseph Asconeguy, Eagle, ID (US); Michael Christopher Oliveira, Escondido, CA (US)

(21) Appl. No.: 19/433,418

(22) Filed: Dec. 26, 2025

Related U.S. Application Data
(60) Provisional application No. 63/740,661, filed on Dec. 31, 2024.

(51) Int. Cl. A61B 17/22

(52) U.S. Cl. CPC A61B 17/22022; A61B 2017/22024; A61B 2017/22038; A61B 2217/007

(57) ABSTRACT

An intravascular lithotripsy device can modify vascular plaque within a blood vessel of a patient by generating sonic waves. The IVL device can include a housing having a distal end, a first insulated wire comprising a first exposed portion adjacent to the distal end of the housing; and a second insulated wire comprising a second exposed portion. The first exposed portion and the second exposed portion can form a spark configured to induce sonic waves configured to travel through the fluid. The IVL device can include a reflective surface positioned proximal to the first exposed portion and the second exposed portion that can reflect the sonic waves to redirect the sonics waves from the proximal direction to a distal direction.

### TECHNICAL FIELD

Any and all applications for which a foreign or domestic priority claim is identified in the Application Data Sheet as filed with the present application are hereby incorporated by reference under 37 CFR 1.57 for all purposes and for all that they contain.

The present disclosure relates generally to intravascular lithotripsy catheters.

### BACKGROUND

An intravascular lithotripsy (IVL) catheter may be used to treat coronary artery disease within the cardiovascular system. IVL catheters can be used to enhance the effectiveness of percutaneous coronary interventions in cases where heavily calcified lesions/plaque present challenges for traditional treatment methods. An IVL catheter can deliver mechanical pressure waves to break up and/or modify calcified lesions, making it easier to dilate an artery during balloon angioplasty and/or stent placement procedures.

### SUMMARY

An intravascular lithotripsy device configured to modify vascular plaque can comprise: a housing configured to hold a fluid, the housing comprising a distal end; a first insulated wire at least partially housed within the housing, wherein the first insulated wire comprises a first exposed portion positioned within the housing; a second insulated wire at least partially housed within the housing, wherein the first insulated wire comprises a second exposed portion positioned within the housing, wherein the first exposed portion and the second exposed portion are configured to form a spark adjacent to the distal end of the housing responsive to an energy differential between the first exposed portion and the second exposed portion exceeding a threshold, wherein the spark is configured to induce sonic waves configured to travel through the fluid; and a reflective surface positioned proximal to the first exposed portion and the second exposed portion, wherein the reflective surface is configured to reflect the sonic waves toward the distal end.

In some implementations, at least one of the first exposed portion or the second exposed portion faces away from the distal end of the housing.

In some implementations, the first exposed portion is at a proximally facing end of the first insulated wire, wherein the second exposed portion is at a proximally facing end of the second insulated wire, wherein the spark is configured to induce the sonic waves to travel proximally through the fluid away from the distal end.

In some implementations, the first exposed portion faces the second exposed portion.

In some implementations, the first exposed portion and the second exposed portion are configured to form the spark across a longitudinal axis of the housing.

In some implementations, the first exposed portion and the second exposed portion are configured to form the spark without forming another spark.

In some implementations, a distal facing portion of the first insulated wire is insulated.

In some implementations, the first insulated wire is curved adjacent to the first exposed portion.

In some implementations, the first insulated wire is curved between 90 degrees and 180 degrees.

In some implementations, the reflective surface is curved.

In some implementations, the reflective surface is configured to focus the sonic waves exterior to the housing distal to the distal end.

In some implementations, the distal end is conical.

In some implementations, the distal end comprises a membrane configured to transfer the sonic waves from the fluid within the housing to a medium outside the housing.

In some implementations, the intravascular lithotripsy device comprises: an irrigation lumen positioned at least partially within the housing and configured to conduct the fluid through the housing toward the distal end to flush a vicinity of a spark gap to inhibit gasses from accumulating in the vicinity of the spark gap and interfering with the sonic waves.

In some implementations, the irrigation lumen is configured to flush the fluid through the housing while the spark forms between the first exposed portion and the second exposed portion.

In some implementations, the irrigation lumen is configured to continuously flush the fluid through the housing.

In some implementations, the housing is configured to conduct the fluid away from the distal end after the fluid has passed from the irrigation lumen through the vicinity of the spark gap.

In some implementations, the intravascular lithotripsy device comprises: a drain lumen positioned at least partially within the housing, wherein the drain lumen is configured to receive the fluid conducted from the irrigation lumen to the distal end of the housing, wherein the drain lumen is configured to conduct the fluid from the distal end of the housing exterior to the housing.

In some implementations, the intravascular lithotripsy device comprises: a guidewire sheath configured to guide the intravascular lithotripsy device along a guidewire, wherein the guidewire sheath is offset from a longitudinal axis of the housing.

In some implementations, the guidewire sheath is positioned outside of the housing.

In some implementations, the guidewire sheath is positioned within the housing.

An intravascular lithotripsy device configured to modify vascular plaque can comprise: a housing configured to hold a fluid, the housing comprising a distal end; a first insulated wire at least partially housed within the housing, wherein the first insulated wire comprises a first exposed portion positioned within the housing; and a second insulated wire at least partially housed within the housing, wherein the first insulated wire comprises a second exposed portion positioned within the housing, wherein the first exposed portion and the second exposed portion are configured to form a spark adjacent to the distal end of the housing responsive to an energy differential between the first exposed portion and the second exposed portion exceeding a threshold, wherein the spark is configured to induce sonic waves configured to travel through the fluid, wherein at least one of the first exposed portion or the second exposed portion faces away from the distal end of the housing.

In some implementations, the intravascular lithotripsy device comprises: a reflective surface positioned proximal to the first exposed portion and the second exposed portion, wherein the reflective surface is configured to reflect the sonic waves toward the distal end.

In some implementations, the first exposed portion is at a proximally facing end of the first insulated wire, wherein the second exposed portion is at a proximally facing end of the second insulated wire, wherein the spark is configured to induce the sonic waves to travel proximally through the fluid away from the distal end.

In some implementations, the first exposed portion faces the second exposed portion.

In some implementations, the first exposed portion and the second exposed portion are configured to form the spark across a longitudinal axis of the housing.

In some implementations, the first exposed portion and the second exposed portion are configured to form the spark independently from forming another spark.

In some implementations, a distal facing portion of the first insulated wire is insulated.

In some implementations, the first insulated wire is curved adjacent to the first exposed portion.

In some implementations, the first insulated wire is curved between 90 degrees and 180 degrees.

In some implementations, the reflective surface is curved.

In some implementations, the reflective surface is configured to focus the sonic waves exterior to the housing distal to the distal end.

In some implementations, the distal end is conical.

In some implementations, the distal end comprises a membrane configured to transfer the sonic waves from the fluid within the housing to a medium outside the housing.

In some implementations, the intravascular lithotripsy device comprises:
an irrigation lumen positioned at least partially within the housing and configured to conduct the fluid through the housing toward the distal end to flush a vicinity of a spark gap to inhibit gasses from accumulating in the vicinity of the spark gap and interfering with the sonic waves.

In some implementations, the irrigation lumen is configured to flush the fluid through the housing while the spark forms between the first exposed portion and the second exposed portion.

In some implementations, the irrigation lumen is configured to continuously flush the fluid through the housing.

In some implementations, the housing is configured to conduct the fluid away from the distal end after the fluid has passed from the irrigation lumen through the vicinity of the spark gap.

In some implementations, the intravascular lithotripsy device comprises: a drain lumen positioned at least partially within the housing, wherein the drain lumen is configured to receive the fluid conducted from the irrigation lumen to the distal end of the housing, wherein the drain lumen is configured to conduct the fluid from the distal end of the housing exterior to the housing.

In some implementations, the intravascular lithotripsy device comprises: a guidewire sheath configured to guide the intravascular lithotripsy device along a guidewire, wherein the guidewire sheath is offset from a longitudinal axis of the housing.

In some implementations, the guidewire sheath is positioned outside of the housing.

In some implementations, the guidewire sheath is positioned within the housing.

Disclosed herein is an intravascular lithotripsy device configured to modify plaque within a blood vessel of a patient by generating sonic waves within the blood vessel. The device can comprise: a housing configured to hold an electrically conductive fluid, the housing comprising a distal end; a plurality of insulated wires extending along a length of the device within the housing, the plurality of insulated wires configured to change voltage responsive to energy conducted through the plurality of insulated wires, the plurality of insulated wires comprising: a first insulated wire comprising a first exposed portion at a proximally facing end of the first insulated wire adjacent to the distal end of the housing; and a second insulated wire comprising a second exposed portion at a proximally facing end of the second insulated wire adjacent to the distal end of the housing, wherein the first exposed portion and the second exposed portion are configured to form a spark adjacent to the distal end of the housing responsive to an energy differential between the first exposed portion and the second exposed portion exceeding a threshold, wherein the spark is configured to induce sonic waves configured to travel through the fluid in a proximal direction away from the distal end. The device can comprise a reflective surface positioned proximal to the first exposed portion and the second exposed portion, the reflective surface configured to reflect the sonic waves to change a direction of travel of the sonics waves from the proximal direction to a distal direction.

In some implementations, a distal facing portion of the plurality of insulated wires is insulated.

In some implementations, the plurality of insulated wires are curved adjacent to the exposed portion.

In some implementations, the plurality of insulated wires are curved 180 degrees.

In some implementations, the reflective surface is curved.

In some implementations, the reflective surface is parabolic.

In some implementations, the reflective surface is configured to focus the sonic waves at a location in the blood vessel distal to the distal end.

In some implementations, the distal end is conical.

In some implementations, the distal end comprises a membrane configured to transfer the sonic waves from the fluid to a blood vessel fluid exterior to the housing.

Disclosed herein is an intravascular lithotripsy device configured to modify plaque within a blood vessel of a patient by generating sonic waves within the blood vessel. The device can comprise: a housing configured to hold a conductive fluid, the housing comprising a distal end; a plurality of insulated wires extending along a length of the device within the housing, the plurality of insulated wires forming an emitter adjacent to the distal end of the housing configured to create a spark to induce sonic waves to travel through the fluid; and an irrigation lumen positioned at least partially within the housing configured to conduct the fluid through the housing toward the distal end to flush a vicinity of the emitter adjacent to the distal end to inhibit gasses from accumulating in the vicinity of the emitter and interfering with the sonic waves, wherein the housing is configured to conduct the fluid exterior to the irrigation lumen away from the distal end after the fluid has passed from the irrigation lumen through the vicinity of the emitter.

In some implementations, the irrigation lumen is configured to flush the fluid through the housing while the plurality of insulated wires form the spark.

In some implementations, the irrigation lumen is configured to continuously flush the fluid through the housing.

In some implementations, the intravascular lithotripsy device comprises a drain lumen positioned at least partially within the housing, wherein the drain lumen is configured to receive the fluid conducted from the irrigation lumen to the distal end of the housing, wherein the drain lumen is configured to conduct the fluid from the distal end of the housing exterior to the housing.

In some implementations, the housing is configured to conduct a larger cross-sectional area of the fluid away from the distal end than the irrigation lumen conducts toward the distal end.

In some implementations, the irrigation lumen is offset from a longitudinal axis of the housing.

In some implementations, the irrigation lumen is closer to one of the plurality of insulated wires than to another.

In some implementations, the irrigation lumen is equidistant to each of the plurality of insulated wires.

Disclosed herein is an intravascular lithotripsy device configured to modify plaque within a blood vessel of a patient by generating sonic waves within the blood vessel. The device can comprise: a housing configured to hold a conductive fluid, the housing comprising a distal end; a plurality of insulated wires extending along a length of the device within the housing, the plurality of insulated wires forming an emitter adjacent to the distal end of the housing configured to create a spark to induce sonic waves to travel through the fluid; and a guidewire sheath configured to guide the intravascular lithotripsy device along a guidewire within the blood vessel, wherein the guidewire sheath extends adjacent to, and offset from, a longitudinal axis of the housing.

In some implementations, the guidewire sheath is positioned outside of the housing.

In some implementations, the guidewire sheath is positioned within the housing.

In some implementations, the guidewire sheath does not extend through the distal end.

In some implementations, the guidewire sheath extends through the distal end.

In some implementations, the distal end extends beyond the guidewire sheath.

In some implementations, the housing extends beyond the guidewire sheath.

In some implementations, the plurality of insulated wires extend beyond the guidewire sheath and form the emitter distal to the guidewire sheath.

In some implementations, the guidewire sheath extends beyond the distal end.

Various combinations of the above and below recited features, embodiments, implementations, and aspects are also disclosed and contemplated by the present disclosure. Additional implementations of the disclosure are described below in reference to the appended claims, which may serve as an additional summary of the disclosure.

### BRIEF DESCRIPTION OF THE DRAWINGS

Various implementations will be described hereinafter with reference to the accompanying drawings. These implementations are illustrated and described by example only, and are not intended to limit the scope of the disclosure. In the drawings, similar elements may have similar reference numerals.

FIG. 1A is a front perspective view of an example IVL device, according to various implementations of the present disclosure.

FIG. 1B is a front perspective view of the IVL device of FIG. 1A with a portion thereof cutaway, according to various implementations of the present disclosure.

FIG. 1C is a bottom perspective view of the IVL device of FIG. 1A with a portion thereof cutaway, according to various implementations of the present disclosure.

FIG. 2 is a top view of the IVL device of FIG. 1A with a portion thereof cutaway, according to various implementations of the present disclosure.

FIG. 3 is a side view of the IVL device of FIG. 1A with a portion thereof cutaway, according to various implementations of the present disclosure.

FIGS. 4A-4C are side cutaway views of the IVL device of FIG. 1A, according to various implementations of the present disclosure.

FIG. 5A is a top cutaway view of the IVL device of FIG. 1A, according to various implementations of the present disclosure.

FIGS. 5B-5C are perspective cutaway views of the IVL device of FIG. 1A, according to various implementations of the present disclosure.

FIG. 5D is a perspective view of an insulated wire with an exposed portion, according to various implementations of the present disclosure.

FIG. 6 is a back cutaway view of the IVL device of FIG. 1A, according to various implementations of the present disclosure.

FIG. 7 is a front cutaway view of the IVL device of FIG. 1A, according to various implementations of the present disclosure.

FIG. 8A is a front perspective view of another example IVL device, according to various implementations of the present disclosure.

FIG. 8B is a front perspective view of the IVL device of FIG. 8A with a portion thereof cutaway, according to various implementations of the present disclosure.

FIG. 9 is a top view of the IVL device of FIG. 8A with a portion thereof cutaway, according to various implementations of the present disclosure.

FIG. 10 is a side view of the IVL device of FIG. 8A with a portion thereof cutaway, according to various implementations of the present disclosure.

FIG. 11 is another side view of the IVL device of FIG. 8A with a portion thereof cutaway, according to various implementations of the present disclosure.

FIGS. 12A-12C are top cutaway views of the IVL device of FIG. 8A, according to various implementations of the present disclosure.

FIG. 12D is a perspective cutaway view of the IVL device of FIG. 8A, according to various implementations of the present disclosure.

FIG. 13 is a side cutaway view of the IVL device of FIG. 8A, according to various implementations of the present disclosure.

FIG. 14 is a rear cutaway view of the IVL device of FIG. 8A, according to various implementations of the present disclosure.

FIG. 15 is a front cutaway view of the IVL device of FIG. 8A, according to various implementations of the present disclosure.

### DETAILED DESCRIPTION

The present disclosure will now be described with reference to the accompanying figures, wherein like numerals may refer to like elements throughout. The following description is merely illustrative in nature and is in no way intended to limit the disclosure, its application, or uses. Furthermore, the devices, systems, and/or methods disclosed herein can include several novel features, no single one of which is solely responsible for its desirable attributes or which is essential to practicing the devices, systems, and/or methods disclosed herein. Additionally, the structures, systems, and/or devices described herein may be embodied as integrated components or as separate components.

Some aspects and/or implementations have been described in connection with the accompanying drawings. The figures are drawn to scale, but such scale is not limiting, since dimensions and proportions other than what are shown are contemplated and are within the scope of the disclosed invention. Distances, angles, etc. are merely illustrative and do not necessarily bear an exact relationship to actual dimensions and layout of the devices illustrated. Components can be added, removed, and/or rearranged. Further, the disclosure herein of any particular feature, aspect, method, property, characteristic, quality, attribute, element, or the like in connection with various embodiments can be used in all other embodiments set forth herein. Additionally, any methods described herein may be practiced using any device suitable for performing the recited steps. Various steps within a method may be executed in different order without altering the principles of the present disclosure.

An intravascular lithotripsy (IVL) device may be used to treat cardiovascular maladies such as coronary artery disease. IVL devices can be used to enhance the effectiveness of percutaneous coronary interventions in cases where vascular plaque, including calcified lesions present challenges for traditional treatment methods. An IVL catheter can deliver sonic waves having mechanical pressure to break up and/or modify plaque, including calcified lesions, making it easier to provide effective treatment such as to dilate an artery during balloon angioplasty and/or stent placement procedures. The IVL devices, techniques, methods shown and/or described herein can treat plaque within peripheral blood vessels, coronary blood vessels, for example to break up the buildup of plaque on blood vessel walls and/or can treat kidney stones in the urinary tract, such as the kidney, ureter, etc.

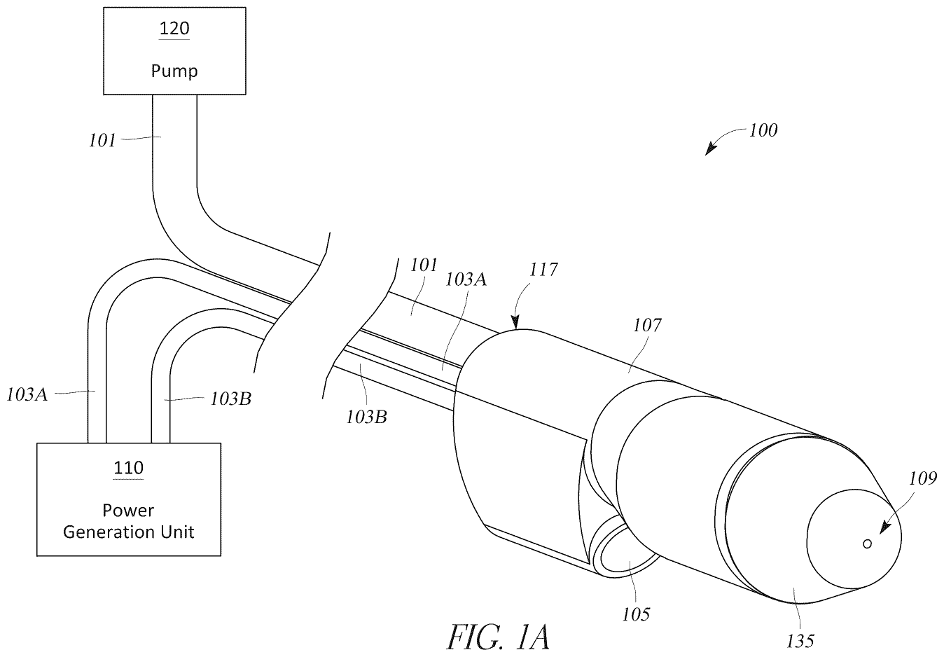

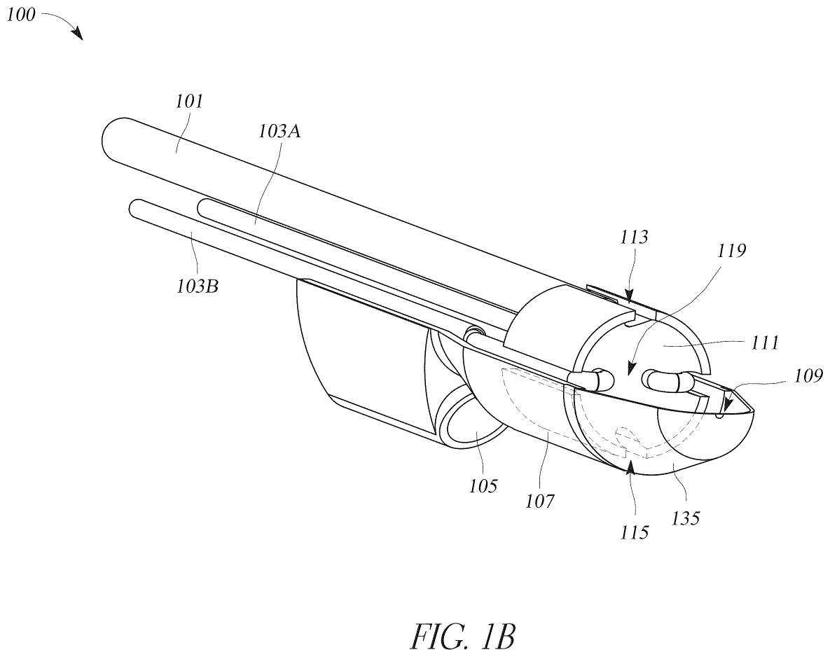

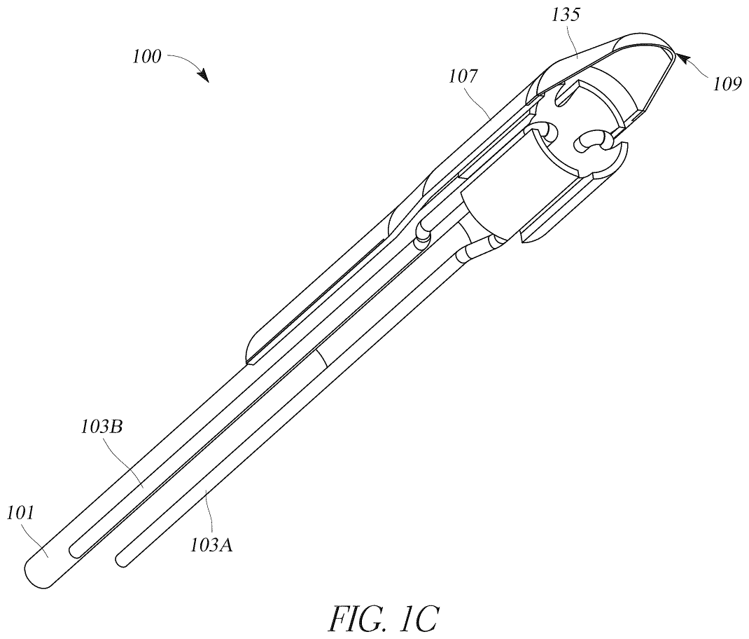

FIG. 1A is a front perspective view of an example IVL device 100. The device 100 includes an irrigation lumen 101, insulated wire 103A, insulated wire 103B, a guidewire sheath 105, a housing 107, a proximal end 117, a membrane 135, and a distal end 109 which can be part of the membrane 135. The membrane 135 can be connected to and form a part of the housing 107. The proximal end 117 may refer to the proximal end of the housing 107. The IVL device 100 can generate sonic waves which can be delivered from the IVL device 100 in a distal direction. The sonic waves can modify calcified deposits in front of the IVL device 100. Thus, the IVL device 100 can advantageously be used when a blood vessel is obstructed by deposits such that the IVL device 100 cannot pass through the blood vessel adjacent to the deposits (thus minimizing the effectiveness of radially delivered sonic waves). In such cases, the IVL device 100 can deliver forward directed sonic waves until the obstruction is cleared and the IVL device 100 can continue to pass through the blood vessel.

The guidewire sheath 105 can conduct the IVL device 100 along a guidewire which can pass through the guidewire sheath 105. The IVL device 100 can travel along the guidewire through the blood vessel to deliver sonic waves toward plaque such as calcified lesions.

The insulated wires 103 (103A and 103B) each can include electrically conductive material, such as metal or metal alloy, such as copper, covered by an electrically insulative material such as plastic or polymer. The insulated wires 103 are electrically connected with an energy generation unit 110 which can generate energy. The energy generation unit 110 can change voltages conducted by the insulated wires 103 and/or amperes conducted along the insulated wires 103. The energy generation unit 110 can induce a voltage differential between insulated wire 103A and insulated wire 103B, which can in turn cause a spark between the insulated wire 103A and insulated wire 103B. In some implementations, the energy generation unit 110 delivers energy to the insulated wires 103 based on sensor data comprising information relating to cardiac activity of a patient. For example, the energy generation unit 110 (and/or one or more hardware processors associated therewith) can receive sensor data from one or more sensors measuring cardiac activity of a patient receiving IVL treatment. The energy generation unit 110 can gate delivery of energy to insulated wires 103 to control when the spark forms based on real-time sensor data indicative of the cardiac cycle of the patient. Thus, the energy generation unit 110 can cause the spark to form at an optimal time (such as during the refractory period of the heat) that will not interfere with the cardiac activity of the patient.

The irrigation lumen 101 is connected to a pump 120. The pump 120 can pressurize fluid and can provide fluid to the irrigation lumen 101 to flow from the pump 120 toward the distal end 109 of the IVL device 100. In some implementations, the pump 120 causes the fluid to continuously flow through the irrigation lumen 101. For example, the pump 120 can maintain a continuous pressure in the fluid to cause the fluid to continuously flow. In some implementations, the pump 120 causes the fluid to periodically flow through the irrigation lumen 101, such as in response to a user request, based on a timer, etc. For example, the pump 120 can pressurize the fluid in discrete increments and/or can gate the fluid from flowing to the irrigation lumen 101 to cause the fluid to flow at a variable rate. Thus, the fluid can flow through the irrigation lumen 101 at a constant rate and/or pressure, or can flow through the irrigation lumen 101 at a variable rate and/or pressure, based on operation of the pump 120. In some implementations, the IVL device 100 may not include a balloon (e.g., a sealed membrane enclosing the insulated wires 103 that changes volume). For example, housing 107 may be rigid or semi-rigid such that housing 107 maintains a substantially fixed volume regardless of fluid or pressure that passes through housing 107. IVL device 100 may be referred to as a non-balloon based device. In some implementations, IVL device 100 may include a balloon attached to, or formed as part of or in place of, housing 107 extending proximally along the IVL device 100.

FIG. 1B is a front perspective view of the IVL device of FIG. 1A with a portion of the housing 107 being cutaway. As shown here, the IVL device 100 includes a reflective surface 111 near the distal end 109, which may also be referred to simply as a “surface”. The insulated wires 103 terminate near the distal end 109 between the reflective surface 111 and the distal end 109. The insulated wires 103 can form a spark gap 119 (which may also be referred to as an emitter) near the distal end 109. Energy differentials between the insulated wires 103 can induce a spark between the insulated wires 103 at the spark gap 119 near the distal end 109. The reflective surface 111 can reflect sonic waves from a spark and redirect them toward the distal end 109. The reflective surface 111 can be at least partially formed of metal, plastic, or any combination thereof. The reflective surface 111 can have one or more properties to facilitate reflecting mechanical energy, such as vibrations, compressions, pressure, and/or waves, such as sonic waves. The properties of the reflective surface 111 may not facilitate reflecting optical radiation such as light. In some implementations, IVL device 100 does not include the reflective surface 111.

The IVL device 100 includes a fluid entrance 113 that can allow fluid to pass from the irrigation lumen 101 toward the distal end 109 and near the spark gap 119. The fluid entrance 113 can be positioned at least partially within the reflective surface 111. For example, the reflective surface 111 can include an opening forming the fluid entrance 113. The IVL device 100 includes a fluid exit 115 that can allow fluid to pass from the spark gap 119 to the interior of the housing 107. The fluid exit 115 can be positioned at least partially within the reflective surface 111. For example, the reflective surface 111 can include an opening forming the fluid exit 115. Thus, fluid can pass (e.g., continuously) over the spark gap 119 where sparks are formed which can inhibit gasses due to electrical discharge from accumulating at the spark gap 119 which may otherwise interfere with sonic wave creation.

The housing 107 can be at least partially formed of a rigid material. The membrane 135 can be sealed to hold fluid within the housing 107. The membrane 135 can be formed a flexible material configured to transfer energy (e.g., sonic waves) therethrough. The housing 107 may not include an expandable material and/or an elastic material, such as a ballon. For example, the membrane 135 may not include a ballon and/or may not expand under pressure. Thus, the volume of the housing 107 and/or membrane 135 may be fixed and/or may not change responsive to differences in pressure between the inside and outside of the housing.

FIG. 1C is a bottom perspective view of the IVL device of FIG. 1A with a portion of the housing 107 being cutaway. As shown, the fluid exit 115 extends from adjacent to the distal end 109 proximally within the housing 107 and conducts fluid proximally from the distal end 109 to the interior of the housing 107 to be flushed at the proximal end 117 of the housing 107.

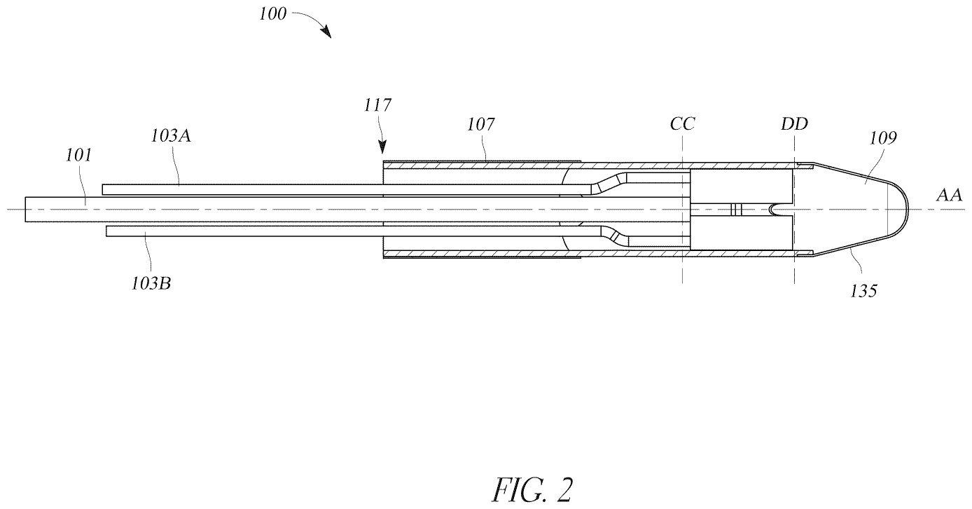

FIG. 2 is a top view of the IVL device 100 with a portion of the housing 107 being cutaway. The insulated wires 103 extend longitudinally along the length of the IVL device 100. The insulated wires 103 can include one or more turns or bends such that at least a portion of the insulated wires 103 are non-parallel (e.g., a distance between the insulated wires 103 is non-uniform). The fluid entrance 113 extends longitudinally within the housing 107 toward the distal end 109 and conducts fluid distally from the irrigation lumen 101 toward the distal end 109 to be flushed over the spark gap 119.

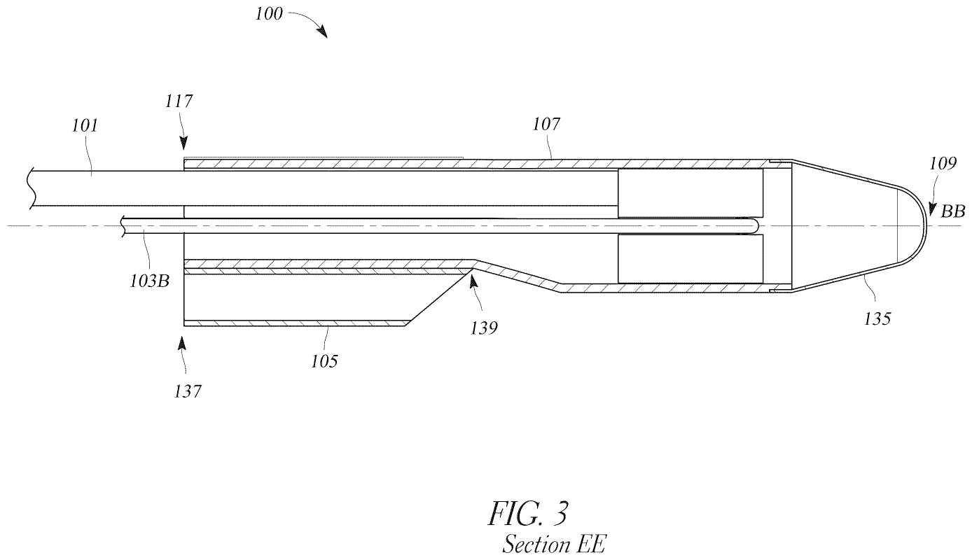

FIG. 3 is a side view of the IVL device 100 with a portion of the housing 107 being cutaway. The irrigation lumen 101 extends longitudinally along the length of the IVL device 100. The irrigation lumen 101 is offset from the central axis of the IVL device 100, denoted here by section plane BB. In some implementations, the irrigation lumen 101 can extend along the central axis.

The guidewire sheath 105 extends longitudinally along the length of the IVL device 100. The guidewire sheath 105 is offset from the central axis of the IVL device 100, denoted here by section plane BB. The guidewire sheath 105 is positioned adjacent to the housing 107, for example, on an exterior surface of the housing 107. In some implementations, the guidewire sheath 105 may be positioned within the housing 107. As shown here, the guidewire sheath 105 includes a proximal side 137 and a distal side 139. In this example, the proximal side 137 of the guidewire sheath 105 is aligned with the proximal end 117 of the housing 107. In various implementations, the proximal side 137 of the guidewire sheath 105 not aligned with the proximal end 117 of the housing 107. For example, the proximal side 137 may be distal or proximal to the proximal end 117. As shown, the distal side 139 of the guidewire sheath 105 can be positioned between the proximal end 117 of the housing and one or more of: the distal end 109 of the housing 107, the membrane 135, the terminus of wire 103A and/or 103B, the spark gap 119, the spark 121, the reflective surface 111, fluid entrance 113, and/or fluid exit 115. The distal end 109 extends beyond the guidewire sheath 105 in a distal direction. For example, the distal end 109 is positioned distal to the distal side 139 of the guidewire sheath 105. The housing 107 extends beyond the guidewire sheath 105 in a distal direction. In this example, the guidewire sheath 105 does not pass through the membrane 135 and/or distal end 109. The insulated wires 103 extend distally beyond the guidewire sheath 105. The insulated wires 103 can form a spark (e.g., at the spark gap 119) distal to the guidewire sheath 105, including the distal side 139 of the guidewire sheath 105. Thus, in this example, the guidewire sheath 105 (and guidewire) do not extend between the spark gap 119 and the membrane 135 and/or distal end 109, thus advantageously allowing sonic waves to travel from the spark 121 toward the membrane 135 and/or distal end 109 without being blocked by the guidewire sheath 105 (or guidewire). The guidewire sheath 105, when offset from the central axis of the IVL device 100, positions the IVL device 100 adjacent to the guidewire running through the guidewire sheath 105. Thus, an offset guidewire sheath 105 can advantageously induce the IVL device 100, including the distal end 109, toward plaque on the blood vessel wall. Proximity to plaque can increase the effectiveness of sonic waves in modifying the plaque.

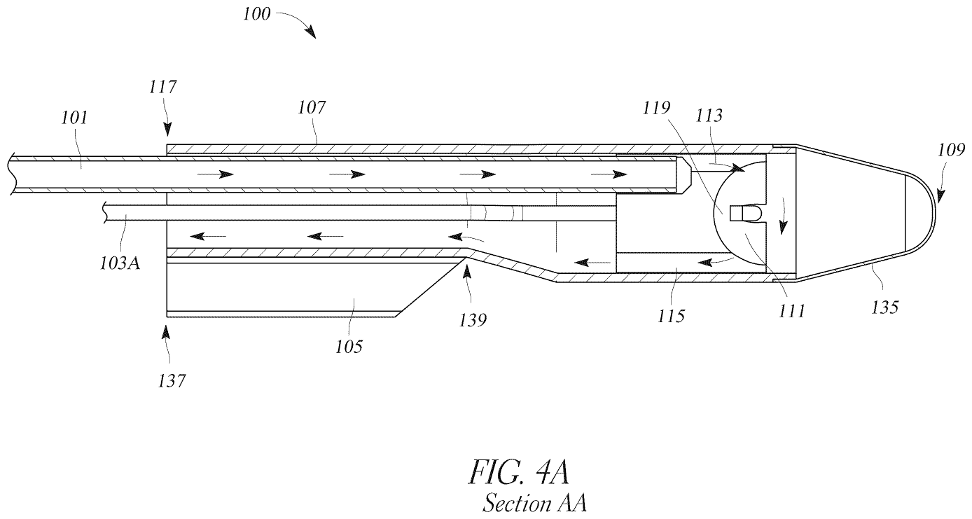

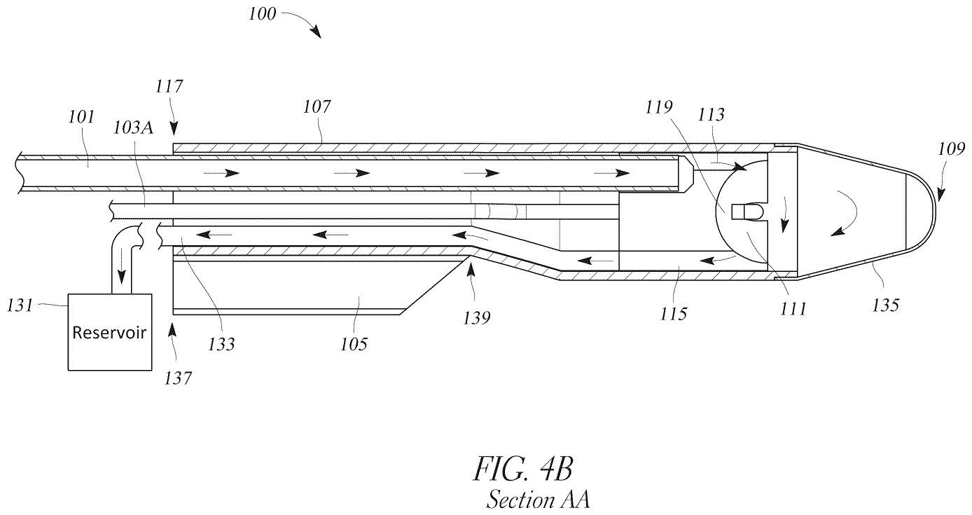

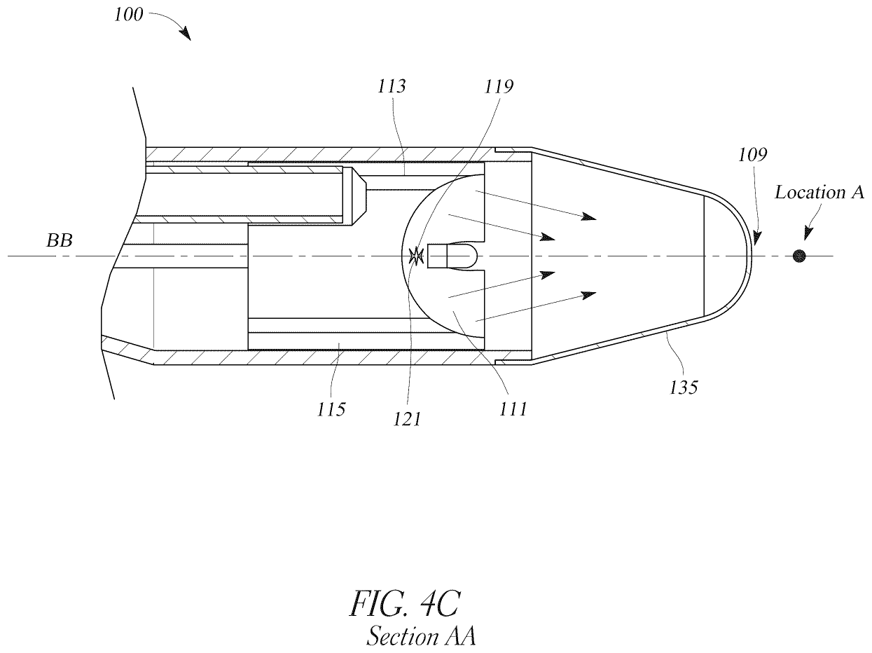

FIG. 4A is a side cutaway view of the IVL device 100 illustrating a path of fluid flow within the IVL device 100 indicated by arrows. The irrigation lumen 101 can conduct fluid toward the distal end 109. The fluid may be an electrically conductive fluid such as a saline solution which can serve as a medium for sonic waves generated by insulated wires 103. The fluid may include contrast to enhance visualization for imaging. The fluid can pass through the fluid entrance 113 toward the distal end 109. The fluid entrance 113 can conduct fluid from the irrigation lumen 101 toward the spark gap 119 where the insulated wires 103 terminate and form sparks. Fluid passing over the spark gap 119 can inhibit gases from accumulating around the spark gap 119 which could inhibit sonic wave formation and/or interfere with sonic waves travelling through the fluid. Fluid can pass in the vicinity of the spark gap 119, including through the spark gap 119, adjacent to the distal end 109, between the spark gap 119 and the distal end 109, between the reflective surface 111 and the spark gap 119, and/or between the reflective surface 111 and the distal end 109. Fluid can pass from the vicinity of the spark gap 119 to the fluid exit 115. The fluid exit 115 can conduct fluid toward the interior of the housing 107 away from the spark gap 119 and/or distal end 109. Fluid within the housing 107 can move proximally along the housing 107 and can be flushed from the housing 107 at the proximal end 117. The fluid can flow from regions of higher pressure to lower pressure and thus can flow from the irrigation lumen 101 to the spark gap 119 near the distal end 109 and then to the interior of the housing 107.

The irrigation lumen 101 has a smaller diameter and/or cross-sectional area than the housing 107. Thus, the irrigation lumen 101 conducts a smaller cross-sectional area of fluid toward the distal end 109 than the housing 107 conducts away from the distal end 109. Accordingly, because the same volume of fluid may travel through the irrigation lumen 101 as travels through the housing 107 (because the system may be closed), the fluid may flow faster through the irrigation lumen 101 toward the distal end 109 than the fluid flows through the housing 107 away from the distal end 109. The housing 107 can have a variable cross-sectional area. For example, the cross-sectional area of the housing 107 can be greater near the distal end 109 than at the proximal end 117. The irrigation lumen 101 may be formed of a rigid or semi-rigid material such that irrigation lumen 101 maintains a substantially fixed volume. Accordingly, the flow rate of fluid passing through lumen 101 can be controlled at a pump.

As shown in FIG. 4B, in some implementations, the IVL device 100 can comprise a drain lumen 133 configured to conduct fluid away from the distal end 109 toward and/or past the proximal end 117 of the housing 107. The drain lumen 133 can be connected to the fluid exit 115. Fluid flowing through the fluid exit 115 can flow into the drain lumen 133. The drain lumen 133 can be connected to a reservoir 131 which can be positioned outside of the housing 107. The reservoir 131 can collect fluid from the drain lumen 133.

FIG. 4C is a side cutaway view of the IVL device 100 illustrating formation of a spark 121 at the spark gap 119. The insulated wires 103 can form the spark 121 adjacent to the distal end 109. The spark 121 can form at a center plane of the housing 107 (indicated here by section plane BB) which can advantageously increase the amount of sonic energy that reaches distal end 109 and/or membrane 135. In some implementations, the IVL device 100 causes only a single spark 121 to form at a time. For example, the insulated wires 103 can form the spark 121 without forming any other sparks. The spark 121 can generate sonic waves. Specifically, the electrical spark 121 can form a plasma bubble that expands and collapses resulting in acoustic pressure (e.g., sonic waves) emanating from the spark 121. Sonic waves can travel away from the distal end 109 in a proximal direction (e.g., to the left with respect to the page) toward the proximal end 117 of the housing 107. The reflective surface 111 can reflect the sonic waves and change the direction of travel of the sonic waves. For example, the sonic waves can reflect off the reflective surface 111 and then travel toward the distal end 109 (after having first travelled in a proximal direction). The reflective surface 111 can be curved, for example, parabolic, spheroidal, etc. In some implementations, the reflective surface 111 is at least partially flat. The reflective surface 111, due in part to its shape, can focus the sonic waves to converge at a same location indicated here as “location A” which may be exterior to the IVL device 100 located distally to the distal end 109 (e.g., in front of the IVL device 100 within the blood vessel). In some implementations, “location A” may be proximal to the distal end 109 within the housing 107. Location A can lie on a center plane of the housing (indicated here by section plane BB) and/or a longitudinal axis or can be offset from the center plane or longitudinal axis. In some implementations, reflective surface 111 can cause sonic waves to diverge from each other increasing the range of directions in which the sonic waves travlel thus increasing the area of treatment. Thus, in some implementations, reflective surface 111 can create more than one “Location A”. Redirecting the sonic waves can increase their effectiveness at modifying plaque, such by constructive interference as sonic waves converge and/or by increasing the directions in which sonic waves travel through divergence. Thus, directing sonic waves proximally to be reflected by the reflective surface 111 can increase efficacy of lithotripsy treatment.

Sonic waves originating from the spark 121 can pass through the membrane 135 at or near the distal end 109 into a medium outside the housing 107, such as blood fluid within a blood vessel and/or plaque. The membrane 135, which can include the distal end 109, can be formed of a material, in whole or in part, that transfers the sonic waves (e.g., acoustic pressure waves) from the interior of the IVL device 100 to the fluid in the blood vessel surrounding the IVL device 100. For example, the membrane 135 may include a flexible membrane that can physically deform from sonic waves and thus facilitate transferring the energy from the sonic waves with minimal energy loss. Sonic wave can pass through all portions of the membrane 135. For example, the guidewire sheath 105 (and/or guidewire) may not pass through the membrane 135 and/or distal end 109 and thus may not block sonic waves from reaching parts of the membrane 135. Thus, in this example, a greater portion of the membrane 135 is exposed to the spark 121 and resulting sonic waves than if the guidewire sheath 105 were positioned between the spark 121 and portions of the membrane 135.

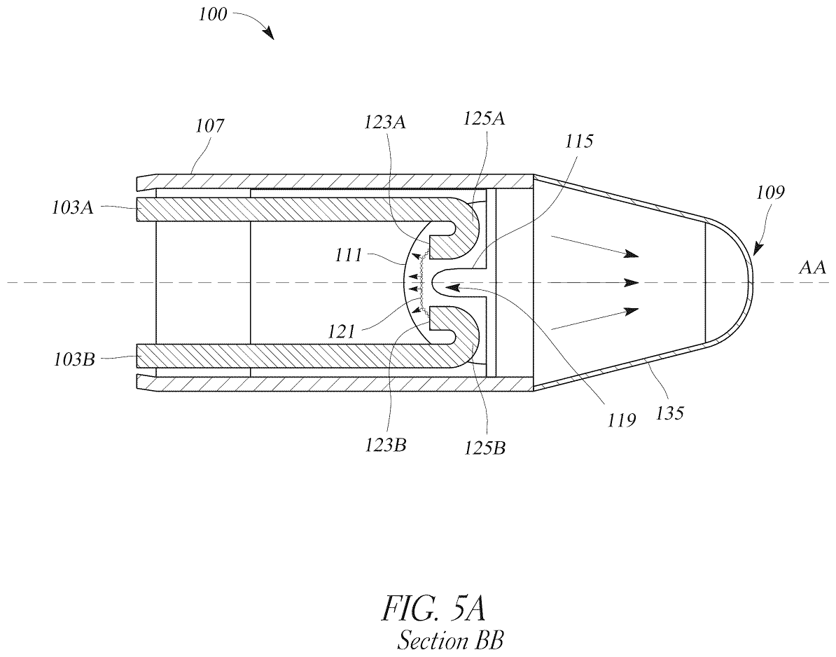

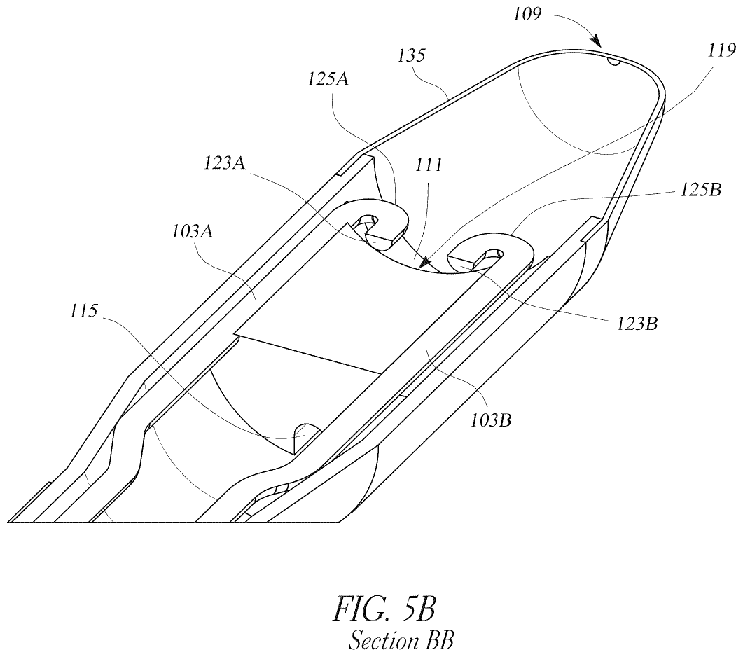

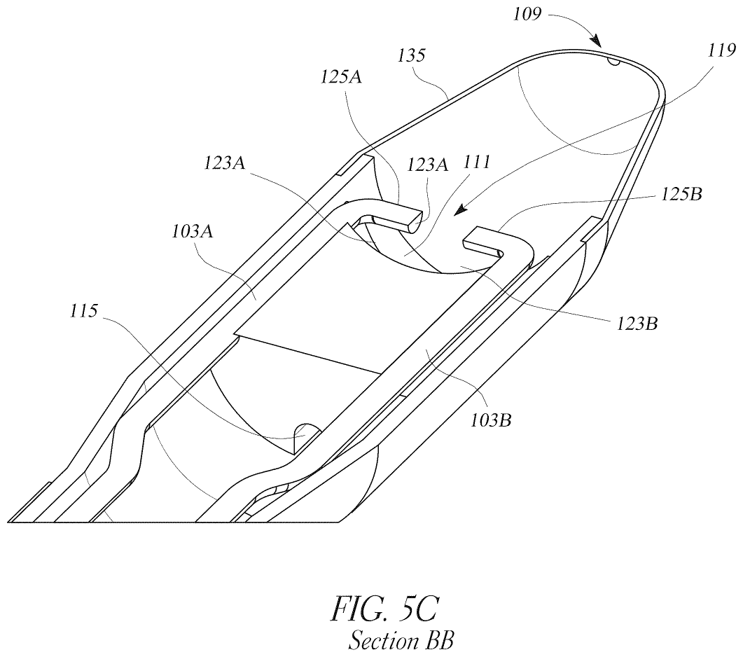

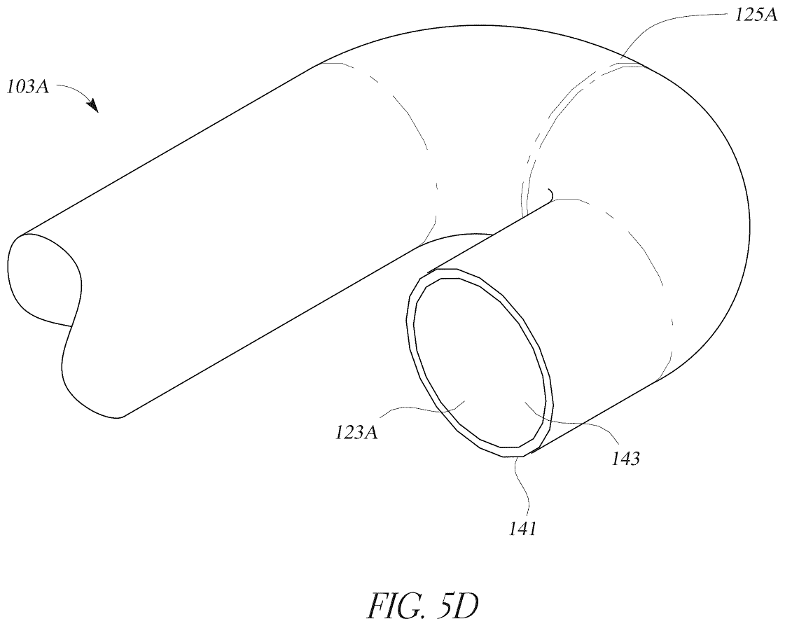

FIG. 5A is a top cutaway view of the IVL device 100 illustrating formation of the spark 121 at the spark gap 119. The insulated wires 103 terminate near the distal end 109. The insulated wires 103 include distal portions 125. Distal portions 125 can be the portions of the insulated wires 103 that are nearest to the distal end 109. Distal portions 125 are curved. In this example, the insulated wires 103 include a 180 degree turn at the distal portions 125 near the exposed portions 123. The insulated wires 103 can extend parallel to section plane AA, turn at the distal portion 125, and then continue extending parallel to section plane AA but in the opposite direction. The insulated wires 103 can be curved near the exposed portions 123 between 45 degrees and 200 degrees, between 90 degrees and 180 degrees, between 135 degrees and 180 degrees, or any value or range of values therebetween. Thus, at least a portion of the insulated wires 103, such as the distal portions 125, can be closer to the distal end 109 of the housing 107 than the exposed portions 123. Thus, the exposed portions 123 may not be the closest part of the insulated wires 103 to the distal end 109. In this example, the exposed portions 123 are perpendicular to a center plane extending through the housing 107 denoted by section plane AA. The insulated wires 103 may not cross section plane AA however the spark gap 119 (and consequently the spark 121) can intersect section plane AA. The distal portions 125 are insulated to inhibit the creation of a spark at the distal portions 125 of the insulated wires 103. In some cases, the distal portions 125 may be uninsulated.

The insulated wires 103 include exposed portions 123 where a portion of electrically conductive material of the insulated wires 103 are exposed. The exposed portions 123 are positioned proximal to the distal portions 125 of the insulated wires 103. The exposed portions 123 are proximally facing and may be referred to as proximal faces or proximally facing exposed portions. The spark gap 119 exists between exposed portion 123A of insulated wire 103A and exposed portion 123B of insulated wire 103B. When an energy differential between exposed portion 123A and exposed portion 123B exceeds a threshold, energy can be conducted between exposed portion 123A and exposed portion 123B resulting in the spark 121. As shown, the spark 121 is formed proximal to the distal portions 125 of the insulated wires 103 and proximal to the exposed portions 123. Because the exposed portions 123A are proximally facing, the spark 121 may induce sonic waves in a proximal direction away from the distal end 109. The spark 121 can follow a generally curved path proximally away from the distal end 109. The reflective surface 111 can reflect proximally travelling sonic waves to redirect their direction of travel in a distal direction toward the distal end 109. The spark 121 crosses a center plane (which may include the longitudinal axis) of the housing 107, indicated here with section plane AA.

FIG. 5B is a perspective cutaway view of the IVL device 100 illustrating the spark gap 119. As shown, the exposed portions 123 are positioned at the ends of the insulated wires 103. The exposed portions 123 face proximally away from the distal end 109. The exposed portions 123 may also be referred to as uninsulated portions, electrodes, or exposed conductive surfaces. The exposed portions 123 may occupy an entire cross-sectional area of the insulated wires 103 or in some cases may occupy less than the entire cross-sectional area of the insulated wires 103.

In some implementations, one or more of the insulated wires 103 can be fixed in place relative to the housing 107 and/or the reflective surface 111. In some implementations, one or more of the insulated wires 103 can move relative to the housing 107 and/or the reflective surface 111. For example, one or more of the insulated wires 103 can move closer to, or further from, the distal end 109. Moving the insulated wire(s) 103 (and consequently the exposed portions 123), can result in sonic waves with different energy leaving the housing 107.

FIG. 5C is a perspective cutaway view of the IVL device 100, according to various implementations. As shown, in various implementations, the exposed portion 123A of insulated wire 103A can face the insulated wire 103B and/or the exposed portion 123B of insulated wire 103B. In this example, the exposed portions 123 of the insulated wires 103 face each other. One or more of the exposed portions 123 may be oriented to face between a proximal direction and a distal direction. One or more of the insulated wires 103 can be curved to between 45 degrees and 135, between 75 degrees and 105 degrees, between 80 degrees and 100 degrees, or any value or range of values therebetween. In this example, the insulated wires 103 are curved to about 90 degrees near their respective exposed portions 123. In this example, the exposed portions 123 are parallel to a center plane extending through the housing 107. In various implementations, one or more of the insulated wires 103 are not curved near their distal ends and/or adjacent to the spark gap 119. In various implementations, one or more of the exposed portions 123 are distal facing. For example, one or more of the exposed portions 123 may face toward the distal end 109. In various implementations, the IVL device 100 may not include the reflective surface 111. As shown here, at least a portion of the insulated wires 103 can extend parallel to section plane AA (shown in FIG. 5A for example), and another portion of the insulated wires 103 (e.g., near the exposed portions 123) can extend non-parallel (for example perpendicular) to the section plane AA.

FIG. 5D is a perspective view of the insulated wire 103A. Insulated wire 103A includes electrically conductive member 143 and electrically insulative member 141. The electrically insulative member 141 encases the electrically conductive member 143. The insulated wire 103A includes exposed portion 123A. At the exposed portion 123A, the electrically conductive member 143 is exposed (e.g., not covered by electrically insulative member 141). The electrically conductive member 143 may be flush with the electrically insulative member 141 at the exposed portion 123A. The exposed portion 123A can be planar. The electrically insulative member 141 can cover the electrically conductive member 143 at the distal portion 125A of the insulated wire 103A. Insulated wire 103B can include the structural and/or operational features of insulated wire 103A.

FIG. 6 is a rear cutaway view of the IVL device 100 showing the interior of the housing 107. The irrigation lumen 101 is physically separated from the insulated wires 103. The irrigation lumen 101 is equidistant to each of the insulated wires 103. The irrigation lumen 101 is physically separated from the housing 107. In some cases, the irrigation lumen may physically contact the insulated wires 103 and/or the housing 107. Fluid flows through the irrigation lumen 101 to the fluid entrance 113 then over the spark gap 119 and then through the fluid exit 115.

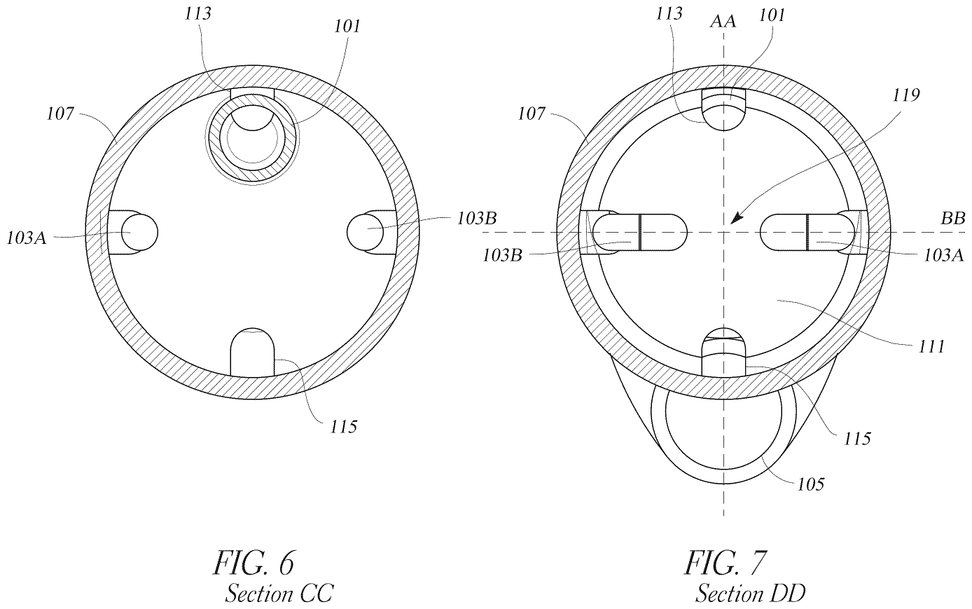

FIG. 7 is a front cutaway view of the IVL device 100 showing the spark gap 119. The fluid entrance 113 is formed in an opening of the reflective surface 111. The fluid exit 115 is formed in an opening of the reflective surface 111. Fluid from the fluid entrance 113 flows over the spark gap 119 and then through the fluid exit 115. Section plane AA is shown extending through a vertical midplane of the housing 107. Section plane BB is shown extending through a horizontal midplane of the housing 107. The longitudinal axis (e.g., center line) of the housing 107 extends along the intersection of section plane BB and section plane AA. Thus, insulated wire 103A and insulated wire 103B can form a spark that spans across at least one midplane (e.g., section plane AA) of the housing 107, lies in at least one midplane (e.g., section plane BB) of housing 107, and/or extends through the longitudinal axis of housing 107. Forming a spark at the center of the housing 107 can facilitate more uniform distribution of sonic energy from the spark and/or housing 107 which can lead to more uniform and/or predictable modification of plaque such as calcified lesions. Offsetting the guidewire sheath 105 from the longitudinal axis (for example by positioning it outside the housing 107 rather than within the housing 107) as shown here can allow the spark to form across one or more midplanes and/or the longitudinal axis.

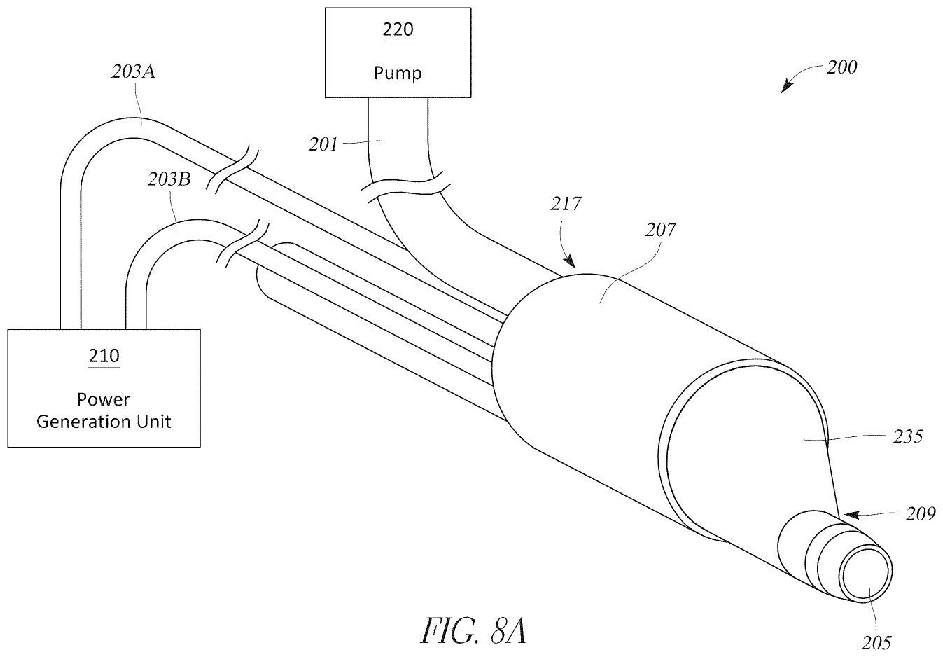

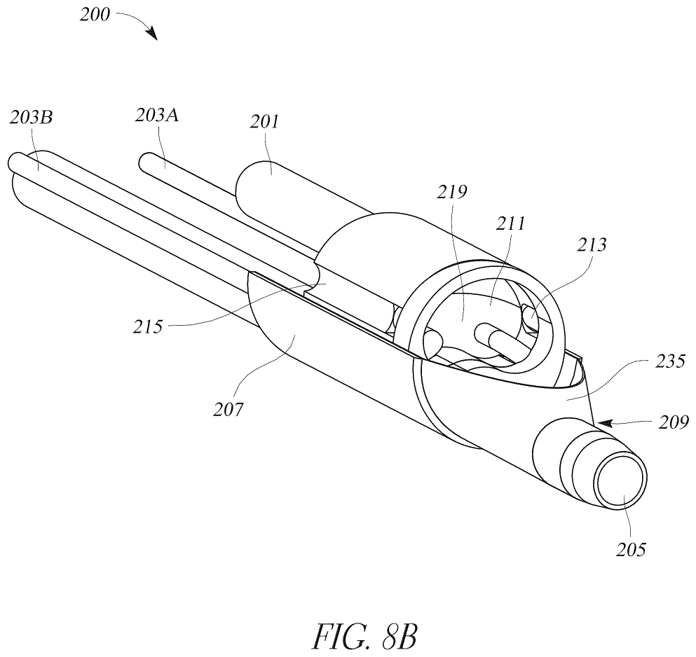

FIG. 8A is a front perspective view of an example IVL device 200. The structural and/or operational features of IVL device 200 and IVL device 100 are not exclusive of each other. The IVL device 200 can include any of the structural and/or operational features of IVL device 100 shown and/or described herein. Likewise, the IVL device 100 can include any of the structural and/or operational features of IVL device 200 shown and/or described herein. The IVL device 200 includes an irrigation lumen 201, insulated wire 203A, insulated wire 203B, a guidewire sheath 205, a housing 207, a proximal end 217, a membrane 235, and a distal end 209. The membrane 235 can be connected to and form a part of the housing 207. The proximal end 217 may refer to the proximal end of the housing 207. The IVL device 200 can generate sonic waves which can be delivered from the IVL device 200 in a distal direction. The sonic waves can modify calcified deposits in front of the IVL device 200. Thus, the IVL device 200 can advantageously be used when a blood vessel is obstructed by deposits such that the IVL device 200 cannot pass through the blood vessel adjacent to the deposits (thus minimizing the effectiveness of radially delivered sonic waves). In such cases, the IVL device 200 can deliver forward directed sonic waves until the obstruction is cleared and the IVL device 200 can continue to pass through the blood vessel.

The guidewire sheath 205 can conduct the IVL device 200 along a guidewire which can pass through the guidewire sheath 205. The IVL device 200 can travel along the guidewire through the blood vessel to deliver sonic waves to plaque.

The insulated wires 203 are electrically connected with an energy generation unit 210 which can generate energy. The energy generation unit 210 can change voltages of the insulated wires 203 and/or amperes conducted along the insulated wires 203. The energy generation unit 210 can induce a voltage differential between insulated wire 203A and insulated wire 203B, which can in turn cause a spark between the insulated wires 203.

The irrigation lumen 201 is connected to a pump 220 which can include any of the structural and/or operational features of pump 120. The pump 220 can pressurize fluid and can provide fluid to the irrigation lumen 201 to flow from the pump 220 toward the distal end 209 of the IVL device 200.

FIG. 8B is a front perspective view of the IVL device of FIG. 8A with a portion of the housing 207 being cutaway. As shown here, the IVL device 200 includes a reflective surface 211 near the distal end 209. The insulated wires 203 terminate near the distal end 209 between the reflective surface 211 and the distal end 209. The insulated wires 203 can form a spark gap 219 (which may also be referred to as an emitter) near the distal end 209. Energy differentials between the insulated wires 203 can induce a spark between the insulated wires 203 at the spark gap 219 near the distal end 209. The reflective surface 211 can reflect sonic waves and redirect them toward the distal end 209.

The IVL device 200 includes a fluid entrance 213 that can allow fluid to pass from the irrigation lumen 201 toward the distal portion and near the spark gap 219. The fluid entrance 213 can be positioned at least partially within, and/or adjacent to, the reflective surface 211. For example, the reflective surface 211 can include an opening forming the fluid entrance 213. The IVL device 200 includes a fluid exit 215 that can allow fluid to pass from the spark gap 219 to the interior of the housing 207. The fluid exit 215 can be positioned at least partially within, and/or adjacent to, the reflective surface 211. For example, the reflective surface 211 can include an opening forming the fluid exit 215. Thus, fluid can pass (e.g., continuously) over the spark gap 219 where sparks are formed which can inhibit gasses due to electrical discharge from accumulating at the spark gap 219 which may otherwise interfere with sonic wave creation.

The housing 207 can be at least partially formed of a rigid material. The membrane 235 can be sealed to hold fluid within the housing 207. The membrane 235 can be formed a flexible material configured to transfer energy (e.g., sonic waves) therethrough. The housing 207 may not include an expandable material and/or an elastic material, such as a ballon. For example, the membrane 235 may not include a ballon and/or may not expand under pressure. Thus, the volume of the housing 207 and/or membrane 235 may be fixed and/or may not change responsive to differences in pressure between the inside and outside of the housing.

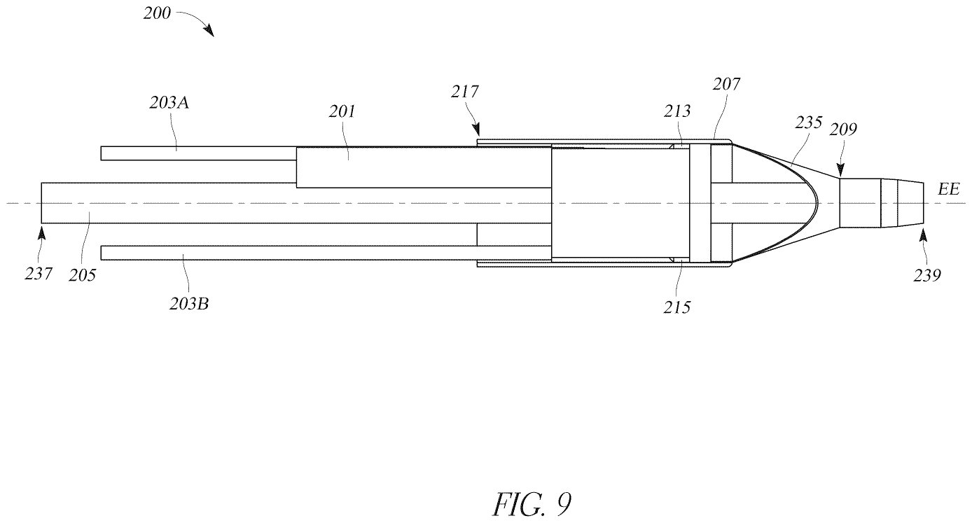

FIG. 9 is a top view of the IVL device 200 with a portion of the housing 207 being cutaway. The insulated wires 203 extend longitudinally along the length of the IVL device 200. The irrigation lumen 201 extends longitudinally along the length of the IVL device 200. The irrigation lumen 201 is offset from the central axis of the IVL device 200, denoted here by section plane FF. In some implementations, the irrigation lumen 201 can extend along the central axis. The fluid entrance 213 extends longitudinally within the housing 207 toward the distal end 209 and conducts fluid distally from the irrigation lumen 201 toward the distal end 209 to be flushed over the spark gap 219. The fluid exit 215 extends longitudinally within the housing 207 away from the distal end 209 and conducts fluid proximally from the distal end 209 to the proximal end of the housing 207.

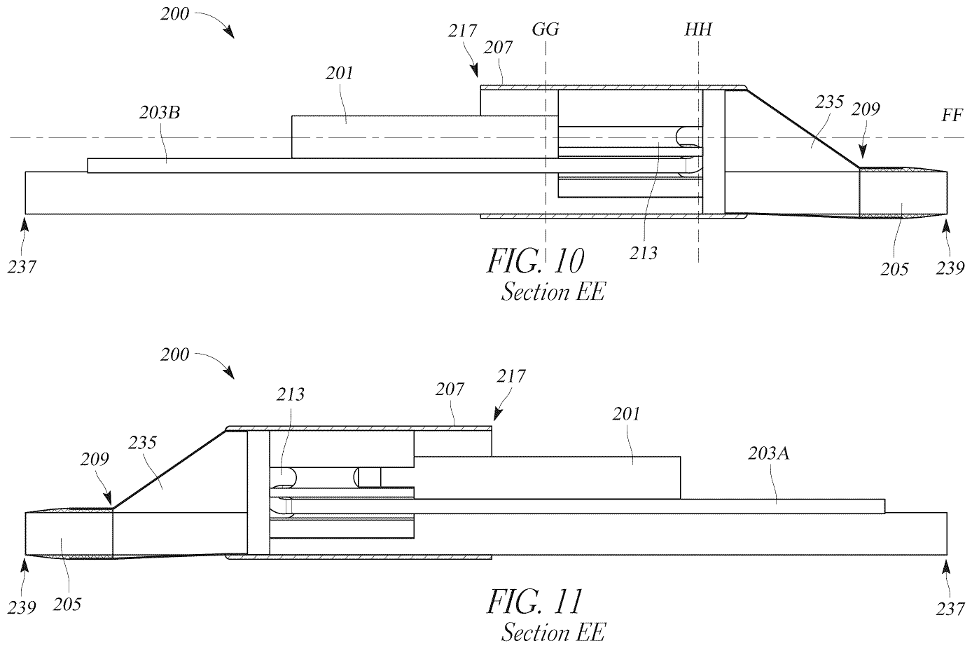

FIGS. 10-11 are side views of the IVL device 200 with a portion of the housing 207 being cutaway. The guidewire sheath 205 extends longitudinally along the length of the IVL device 200. The guidewire sheath 205 is offset from the central axis of the IVL device 200, denoted here by section plane FF. The guidewire sheath 205, when offset from the central axis of the IVL device 200, positions the IVL device 200 adjacent to the guidewire running through the guidewire sheath 205. Thus, an offset guidewire sheath 205 can advantageously induce the IVL device 200, including the membrane 235 and/or distal end 209, toward plaque on the blood vessel wall. Proximity to plaque can increase the effectiveness of sonic waves in modifying the plaque. The guidewire sheath 205 extends through the housing 207. The guidewire sheath 205 can extend beyond the distal end 209 and/or the housing 207 in a distal direction. The guidewire sheath 205 extends beyond the insulated wires 203 in a distal direction and thus also beyond a spark formed by insulated wires 203. One or more of the spark gap 219, the exposed portions 223, the membrane 235, and/or the distal end 209 can be positioned between the proximal side 237 of the guidewire sheath 205 and the distal side 239 of the guidewire sheath 205. The membrane 235 can seal against the guidewire sheath 205. The guidewire sheath 205 can extend through the membrane 235. Positioning the guidewire sheath 205 to extend through the housing 207 and/or the membrane 235 can allow for greater navigational control when advancing the IVL device 200 along the guidewire at least because the center of mass of the housing 207 will be closer to the axis of rotation of the guidewire sheath 205. The distal end 209 of the housing 207 can represent the most distal location to which conductive fluid held by the housing 207 can reach. For example, conductive fluid within the housing 207 may not extend distally past the distal end 209.

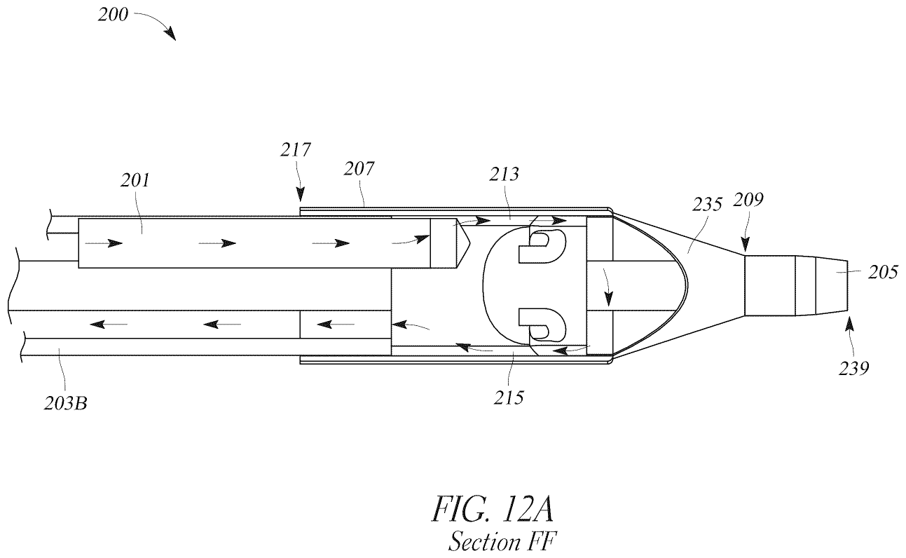

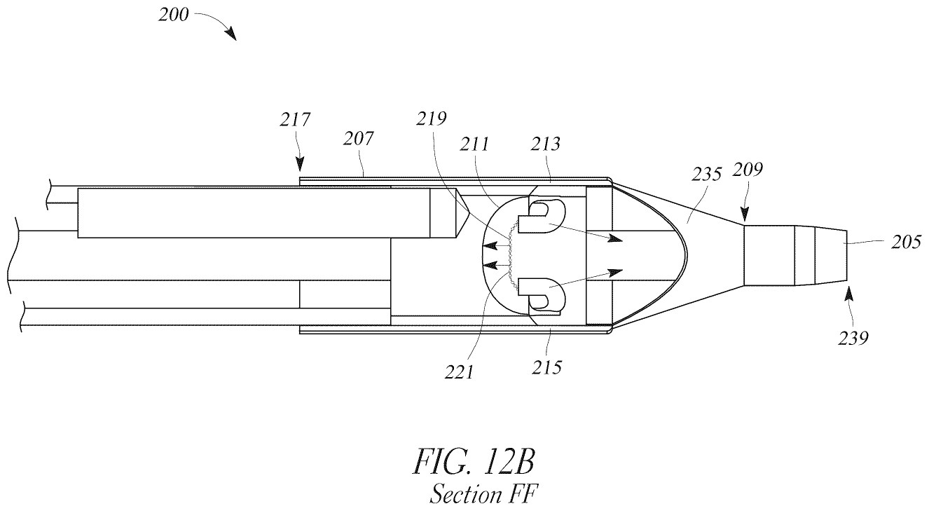

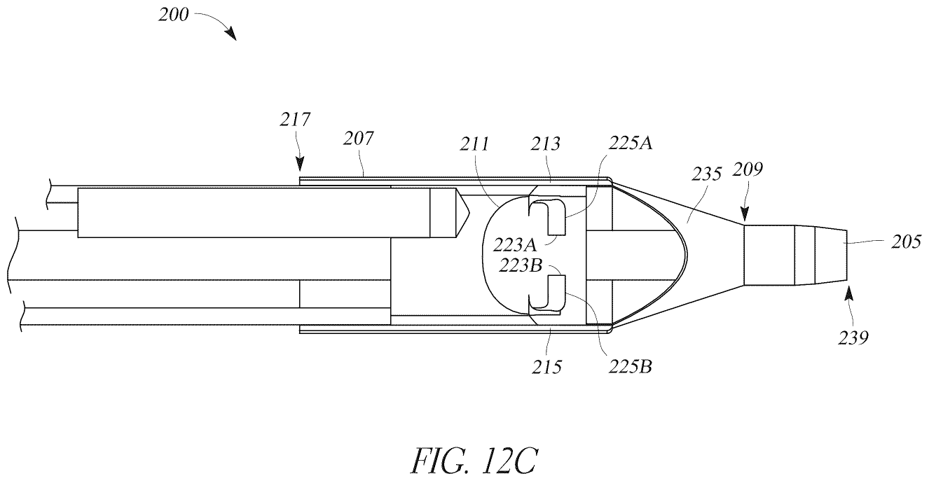

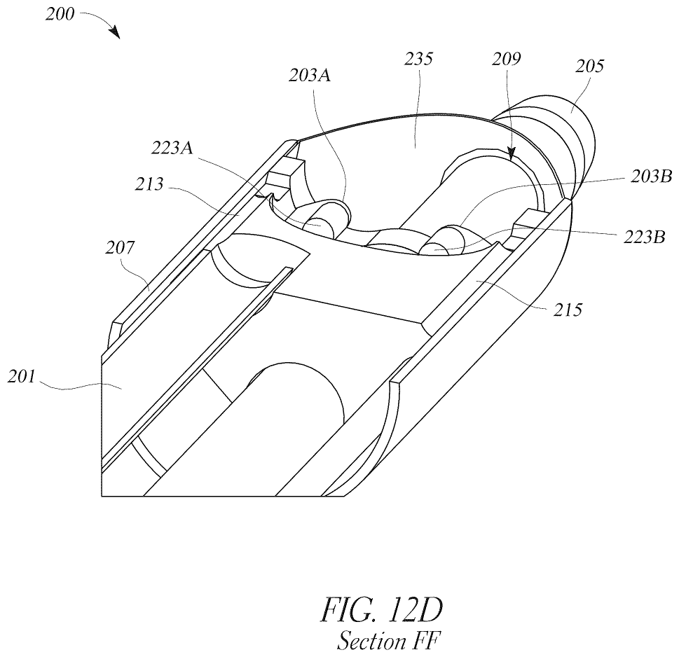

FIG. 12A is a top cutaway view of the IVL device 200 illustrating a path of fluid flow within the IVL device 200 indicated by arrows. The irrigation lumen 201 can conduct fluid toward the distal end 209. The fluid can pass through the fluid entrance 213 toward the distal end 209. The fluid entrance 213 can conduct fluid from the irrigation lumen 201 toward the spark gap 219 where the insulated wires 203 terminate and form sparks. Fluid passing over the spark gap 219 can inhibit gases from accumulating around the spark gap 219 which could inhibit sonic wave formation. Fluid can pass from the spark gap 219 adjacent to the distal end 209 to the fluid exit 215. The fluid exit 215 can conduct fluid toward the interior of the housing 207 away from the spark gap 219 and/or distal end 209. Fluid within the housing 207 can move proximally along the housing 207 and can be flushed from the housing 207 at the proximal end 217. The fluid can flow from regions of higher pressure to lower pressure and thus can flow from the irrigation lumen 201 to the spark gap 219 near the distal end 209 and then to the interior of the housing 207.

The irrigation lumen 201 has a smaller diameter and/or cross-sectional area than the housing 207. Thus, the irrigation lumen 201 conducts a smaller cross-sectional area of fluid toward the distal end 209 than the housing 207 conducts away from the distal end 209. Accordingly, because the same volume of fluid may travel through the irrigation lumen 201 as travels through the housing 207 (because the system may be closed), the fluid may flow faster through the irrigation lumen 201 toward the distal end 209 than the fluid flows through the housing 207 away from the distal end 209. The housing 207 has a uniform cross-sectional area from the proximal end 217 to the distal end 209.

FIG. 12B is a top cutaway view of the IVL device 200 illustrating formation of a spark 221 at the spark gap 219. The insulated wires 203 can form the spark 221 adjacent to the distal end 209. The spark 221 can generate sonic waves. Specifically, the electrical spark 221 can form a plasma bubble that expands and collapses resulting in acoustic pressure (e.g., sonic waves) emanating from the spark 221. Sonic waves can travel away from the distal end 209 in a proximal direction (e.g., to the left with respect to the page) toward the proximal end 217 of the housing 207. The reflective surface 211 can reflect the sonic waves and change the direction of travel of the sonic waves. For example, the sonic waves can reflect off the reflective surface 211 and then travel toward the distal end 209 (after having first travelled in a proximal direction). The reflective surface 211 can be curved, for example, parabolic, spheroidal, etc. In some aspects, the reflective surface 211 may be non-curved and/or non-parabolic. The curved shape of the reflective surface 211 can focus the sonic waves to intersect at a same location which may be exterior to the IVL device 200 located distally to the distal end 209 (e.g., in front of the IVL device 200 within the blood vessel) or in some cases may be within the distal end 209. Focusing the sonic waves can amplify their energy as they constructively interfere with each other which can increase their effectiveness at modifying plaque.

Sonic waves originating from the spark 221 can pass through the membrane 235 into the fluid of the blood vessel. The membrane 235 can be formed of a material, in whole or in part, that transfers the sonic waves (e.g., acoustic pressure waves) from the interior of the IVL device 200 to the fluid in the blood vessel surrounding the IVL device 200. For example, the membrane 235 can physically deform from sonic waves and thus transfer the energy from the sonic waves with minimal energy loss.

The insulated wires 203 terminate near the distal end 209. The insulated wires 203A, 203B include distal portions 225A, 225B, respectively. Distal portions 225 can be the portions of the insulated wires 203 that are nearest to the distal end 209. Distal portions 225 are curved. In this example, the insulated wires 203 include a 180 degree turn at the distal portions 225. The distal portions 225 are insulated to inhibit the creation of a spark at the distal portions 225 of the insulated wires 203. In some cases, the distal portions 225 may be uninsulated.

FIG. 12C is a perspective cutaway view of the IVL device 200, according to various implementations. As shown, in various implementations, the exposed portion 223A of insulated wire 203A can face the insulated wire 203B and/or the exposed portion 223B of insulated wire 203B. In this example, the exposed portions 223 of the insulated wires 203 face each other. One or more of the exposed portions 223 may be oriented to face between a proximal direction and a distal direction. One or more of the insulated wires 203 can be curved to between 45 degrees and 135, between 75 degrees and 105 degrees, between 80 degrees and 100 degrees, or any value or range of values therebetween. In this example, the insulated wires 203 are curved to about 90 degrees near their respective exposed portions 223. In this example, the exposed portions 223 are parallel to a center plane extending through the housing 207. In various implementations, one or more of the insulated wires 203 are not curved near their distal ends and/or adjacent to the spark gap 219. In various implementations, one or more of the exposed portions 223 are distal facing. For example, one or more of the exposed portions 223 may face toward the distal end 209. In various implementations, the IVL device 200 may not include the reflective surface 211

FIG. 12D is a perspective cutaway view of the IVL device 200 illustrating the spark gap 219. The insulated wires 203A, 203B include exposed portions 223A, 223B, respectively. The exposed portions 223 are positioned proximal to the distal portions 225 of the respective insulated wires 203. The exposed portions 223 are proximally facing and may be referred to as proximal faces or proximally facing exposed portions. The spark gap 219 exists between exposed portion 223A of insulated wire 203A and exposed portion 223B of insulated wire 203B. When an energy differential between exposed portion 223A and exposed portion 223B exceeds a threshold, energy can be conducted between exposed portion 223A and exposed portion 223B resulting in the spark 221. As shown in FIG. 12B, the spark 221 is formed proximal to the distal portions 225 of the insulated wires 203. Because the exposed portions 223A are proximally facing, the spark 221 may induce sonic waves in a proximal direction away from the distal end 209. The spark 221 can follow a generally curved path proximally away from the distal end 209. The reflective surface 211 can reflect proximally travelling sonic waves to redirect their direction of travel in a distal direction toward the distal end 209.

As shown, the exposed portions 223 are positioned at the ends of the insulated wires 203. The exposed portions 223 face proximally away from the distal end 209. The exposed portions 223 may also be referred to as uninsulated portions, electrodes or exposed conductive surfaces. The exposed portions 223 may occupy an entire cross-sectional area of the insulated wires 203 or in some cases may occupy less than the entire cross-sectional area of the insulated wires 203.

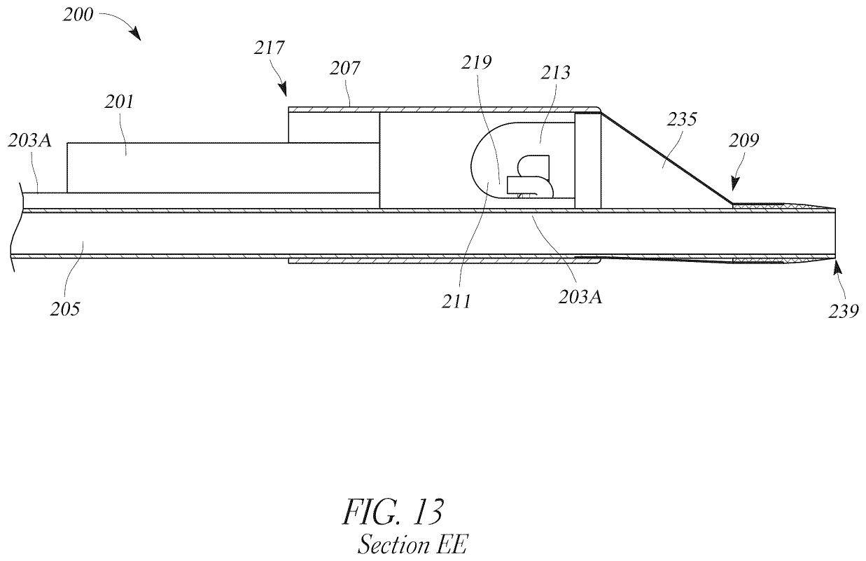

FIG. 13 is a side cutaway view of the IVL device 200. The reflective surface 211 may be non-spheroidal.

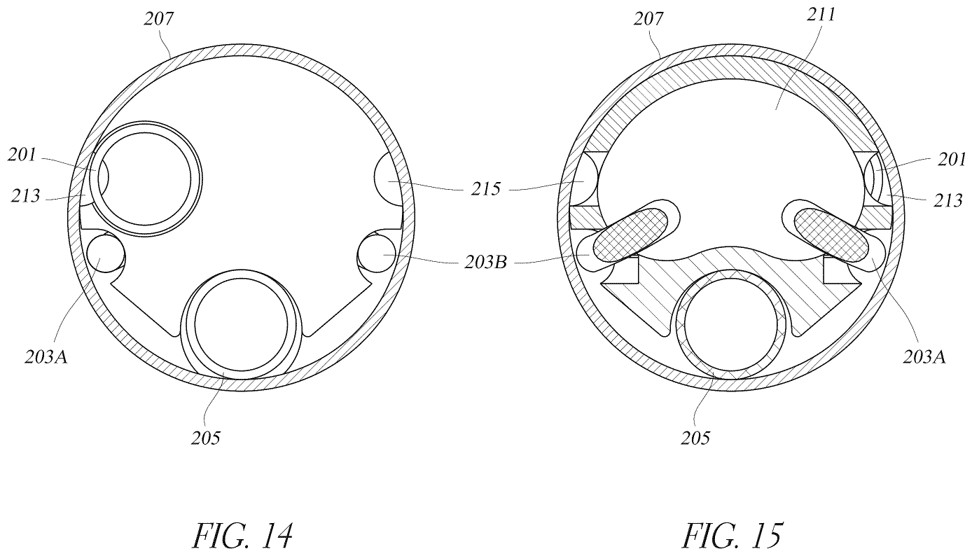

FIG. 14 is a rear cutaway view of the IVL device 200 showing the interior of the housing 207. The irrigation lumen 201 is physically separated from the insulated wires 203. The irrigation lumen 201 is closer to insulated wire 203A than to insulated wire 203B. The irrigation lumen 201 is offset from a longitudinal axis of the IVL device 200 extending through the center of the housing 207. The irrigation lumen 201 is physically separated from the housing 207. In some cases, the irrigation lumen may physically contact the insulated wires 203 and/or the housing 207. Fluid flows through the irrigation lumen 201 to the fluid entrance 213 then over the spark gap 219 and then through the fluid exit 215.

FIG. 15 is a front cutaway view of the IVL device 200 showing the spark gap 219. The fluid entrance 213 is adjacent to the reflective surface 211. The fluid exit 215 is adjacent to the reflective surface 211. Fluid from the fluid entrance 213 flows over the spark gap 219 and then through the fluid exit 215.

### Additional Considerations

Although certain implementations and examples have been described herein, it will be understood by those skilled in the art that many aspects of the systems and devices shown and described in the present disclosure may be differently combined and/or modified to form still further implementations or acceptable examples. All such modifications and variations are intended to be included herein within the scope of this disclosure. A wide variety of designs and approaches are possible. No feature, structure, or step disclosed herein is essential or indispensable. The various features and processes described herein may be used independently of one another, or may be combined in various ways. For example, elements may be added to, removed from, or rearranged compared to the disclosed example implementations. All possible combinations and sub-combinations are intended to fall within the scope of this disclosure.

Any methods and processes described herein are not limited to any particular sequence, and the blocks or states relating thereto can be performed in other sequences that are appropriate. For example, described blocks or states may be performed in an order other than that specifically disclosed, or multiple blocks or states may be combined in a single block or state, or certain method or process blocks may be omitted, or certain blocks or states may be performed in a reverse order from what is shown and/or described. The example blocks or states may be performed in serial, in parallel, or in some other manner. Blocks or states may be added to or removed from the disclosed example implementations.

The methods disclosed herein may include certain actions taken by a practitioner; however, they can also include any third-party instruction of those actions, either expressly or by implication.

The methods and tasks described herein may be performed and fully automated by a computer system. The computer system may, in some cases, include multiple distinct computers or computing devices (e.g., physical servers, workstations, storage arrays, cloud computing resources, etc.) that communicate and interoperate over a network to perform the described functions. Each such computing device typically includes a processor (or multiple processors) that executes program instructions or modules stored in a memory or other non-transitory computer-readable storage medium or device (e.g., solid state storage devices, disk drives, etc.). The various functions disclosed herein may be embodied in such program instructions, and/or may be implemented in application-specific circuitry (e.g., ASICs or FPGAs) of the computer system. Where the computer system includes multiple computing devices, these devices may, but need not, be co-located. The results of the disclosed methods and tasks may be persistently stored by transforming physical storage devices, such as solid state memory chips and/or magnetic disks, into a different state. The computer system may be a cloud-based computing system whose processing resources are shared by multiple distinct entities or other users. The systems and modules may also be transmitted as generated data signals (for example, as part of a carrier wave or other analog or digital propagated signal) on a variety of computer-readable transmission mediums, including wireless-based and wired/cable-based mediums, and may take a variety of forms (for example, as part of a single or multiplexed analog signal, or as multiple discrete digital packets or frames).

Many other variations than those described herein will be apparent from this disclosure. For example, depending on the implementation, certain acts, events, or functions of any of the algorithms described herein can be performed in a different sequence, can be added, merged, or left out altogether (for example, not all described acts or events are necessary for the practice of the algorithms). Moreover, in certain implementations, acts or events can be performed concurrently, for example, through multi-threaded processing, interrupt processing, or multiple processors or processor cores or on other parallel architectures, rather than sequentially. In addition, different tasks or processes can be performed by different machines and/or computing systems that can function together.

Various illustrative logical blocks, modules, routines, and algorithm steps that may be described in connection with the disclosure herein can be implemented as electronic hardware (e.g., ASICs or FPGA devices), computer software that runs on computer hardware, or combinations of both. Various illustrative components, blocks, and steps may be described herein generally in terms of their functionality. Whether such functionality is implemented as specialized hardware versus software running on general-purpose hardware depends upon the particular application and design constraints imposed on the overall system. The described functionality can be implemented in varying ways for each particular application, but such implementation decisions should not be interpreted as causing a departure from the scope of the disclosure.

Moreover, various illustrative logical blocks and modules that may be described in connection with the implementations disclosed herein can be implemented or performed by a machine, such as a general purpose processor, a digital signal processor (“DSP”), an application specific integrated circuit (“ASIC”), a field programmable gate array (“FPGA”) or other programmable logic device, discrete gate or transistor logic, discrete hardware components, or any combination thereof designed to perform the functions described herein. A general purpose processor can be a microprocessor, controller, microcontroller, or state machine, combinations of the same, or the like. A processor can include electrical circuitry configured to process computer-executable instructions. A processor can include an FPGA or other programmable devices that performs logic operations without processing computer-executable instructions. A processor can also be implemented as a combination of computing devices, for example, a combination of a DSP and a microprocessor, a plurality of microprocessors, one or more microprocessors in conjunction with a DSP core, or any other such configuration. Although described herein primarily with respect to digital technology, a processor may also include primarily analog components. For example, some, or all, of the signal processing algorithms described herein may be implemented in analog circuitry or mixed analog and digital circuitry. A computing environment can include any type of computer system, including, but not limited to, a computer system based on a microprocessor, a mainframe computer, a digital signal processor, a portable computing device, a device controller, or a computational engine within an appliance, to name a few.

The elements of any method, process, routine, or algorithm described in connection with the disclosure herein can be embodied directly in hardware, in a software module executed by a processor, or in a combination of the two. A software module can reside in RAM memory, flash memory, ROM memory, EPROM memory, EEPROM memory, registers, hard disk, a removable disk, a CD-ROM, or any other form of a non-transitory computer-readable storage medium. An exemplary storage medium can be coupled to the processor such that the processor can read information from, and write information to, the storage medium. In the alternative, the storage medium can be integral to the processor. The storage medium can be volatile or nonvolatile. The processor and the storage medium can reside in an ASIC. The ASIC can reside in a user terminal. In the alternative, the processor and the storage medium can reside as discrete components in a user terminal.

Conditional language used herein, such as, among others, “can,” “could,” “might,” “may,” “e.g.,” and the like, unless specifically stated otherwise, or otherwise understood within the context as used, is generally intended to convey that certain features, elements, and/or steps are optional. Thus, such conditional language is not generally intended to imply that features, elements, and/or steps are in any way required or that one or more embodiments necessarily include logic for deciding, with or without other input or prompting, whether these features, elements, and/or steps are included or are to be always performed. The terms “comprising,” “including,” “having,” and the like are synonymous and are used inclusively, in an open-ended fashion, and do not exclude additional elements, features, acts, operations, and so forth. Also, the term “or” is used in its inclusive sense (and not in its exclusive sense) so that when used, for example, to connect a list of elements, the term “or” means one, some, or all of the elements in the list. Further, the term “each,” as used herein, in addition to having its ordinary meaning, can mean any subset of a set of elements to which the term “each” is applied.

Conjunctive language such as the phrase “at least one of X, Y, and Z,” unless specifically stated otherwise, is otherwise understood with the context as used in general to convey that an item, term, etc. may be either X, Y, or Z. Thus, such conjunctive language is not generally intended to imply that certain embodiments require the presence of at least one of X, at least one of Y, and at least one of Z.

Language of degree used herein, such as the terms “approximately,” “about,” “generally,” and “substantially” as used herein represent a value, amount, or characteristic close to the stated value, amount, or characteristic that still performs a desired function or achieves a desired result. For example, the terms “approximately”, “about”, “generally,” and “substantially” may refer to an amount that is within less than 10% of, within less than 5% of, within less than 1% of, within less than 0.1% of, and within less than 0.01% of the stated amount. As another example, in certain embodiments, the terms “generally parallel” and “substantially parallel” refer to a value, amount, or characteristic that departs from exactly parallel by less than or equal to 10 degrees, 5 degrees, 3 degrees, or 1 degree. As another example, in certain embodiments, the terms “generally perpendicular” and “substantially perpendicular” refer to a value, amount, or characteristic that departs from exactly perpendicular by less than or equal to 10 degrees, 5 degrees, 3 degrees, or 1 degree.

As used herein, “real-time” or “substantial real-time” may refer to events (e.g., receiving, processing, transmitting, displaying etc.) that occur at a same time as each other, during a same time as each other, or overlap in time with each other. “Real-time” may refer to events that occur at distinct or non-overlapping times the difference between which is imperceptible and/or inconsequential to humans such as delays arising from electrical conduction or transmission. A human may perceive real-time events as occurring simultaneously, regardless of whether the real-time events occur at an exact same time. As a non-limiting example, “real-time” may refer to events that occur within a time frame of each other that is on the order of milliseconds, seconds, tens of seconds, or minutes. For example, “real-time” may refer to events that occur within a time frame of less than 1 minute, less than 30 seconds, less than 10 seconds, less than 1 second, less than 0.05 seconds, less than 0.01 seconds, less than 0.005 seconds, less than 0.001 seconds, etc.

Unless otherwise explicitly stated, articles such as “a” or “an” should generally be interpreted to include one or more described items. Accordingly, phrases such as “a device configured to” are intended to include one or more recited devices. Such one or more recited devices can also be collectively configured to carry out the stated recitations. For example, “a processor configured to carry out recitations A, B and C” can include a first processor configured to carry out recitation A working in conjunction with a second processor configured to carry out recitations B and C.

As used herein, “system,” “instrument,” “apparatus,” and “device” generally encompass both the hardware (for example, mechanical and electronic) and, in some implementations, associated software (for example, specialized computer programs for operational control) components.

It should be emphasized that many variations and modifications may be made to the herein-described implementations, the elements of which are to be understood as being among other acceptable examples. All such modifications and variations are intended to be included herein within the scope of this disclosure. Any section headings used herein are merely provided to enhance readability and are not intended to limit the scope of the implementations disclosed in a particular section to the features or elements disclosed in that section. The foregoing description details certain implementations. It will be appreciated, however, that no matter how detailed the foregoing appears in text, the systems and methods can be practiced in many ways. As is also stated herein, it should be noted that the use of particular terminology when describing certain features or aspects of the systems and methods should not be taken to imply that the terminology is being re-defined herein to be restricted to including any specific characteristics of the features or aspects of the systems and methods with which that terminology is associated.

Those of skill in the art would understand that information, messages, and signals may be represented using any of a variety of different technologies and techniques. For example, data, instructions, commands, information, signals, bits, symbols, and chips that may be referenced throughout the above description may be represented by voltages, currents, electromagnetic waves, magnetic fields or particles, optical fields or particles, or any combination thereof.

While the above detailed description has shown, described, and pointed out novel features, it can be understood that various omissions, substitutions, and changes in the form and details of the devices or algorithms illustrated can be made without departing from the spirit of the disclosure. As can be recognized, certain portions of the description herein can be embodied within a form that does not provide all of the features and benefits set forth herein, as some features can be used or practiced separately from others. The scope of certain embodiments disclosed herein is indicated by the appended claims rather than by the foregoing description. All changes which come within the meaning and range of equivalency of the claims are to be embraced within their scope.

What is claimed is:

1. An intravascular lithotripsy device configured to modify vascular plaque, comprising:
a housing configured to hold a fluid, the housing comprising a distal end;
a first insulated wire at least partially housed within the housing, wherein the first insulated wire comprises a first exposed portion positioned within the housing;
a second insulated wire at least partially housed within the housing, wherein the first insulated wire comprises a second exposed portion positioned within the housing, wherein the first exposed portion and the second exposed portion are configured to form a spark adjacent to the distal end of the housing responsive to an energy differential between the first exposed portion and the second exposed portion exceeding a threshold, wherein the spark is configured to induce sonic waves configured to travel through the fluid; and
a reflective surface positioned proximal to the first exposed portion and the second exposed portion, wherein the reflective surface is configured to reflect the sonic waves toward the distal end.

2. The intravascular lithotripsy device of claim 1, wherein at least one of the first exposed portion or the second exposed portion faces away from the distal end of the housing.

3. The intravascular lithotripsy device of claim 1, wherein the first exposed portion and the second exposed portion are configured to form the spark across a longitudinal axis of the housing.

4. The intravascular lithotripsy device of claim 1, wherein the first insulated wire is curved adjacent to the first exposed portion.

5. The intravascular lithotripsy device of claim 1, wherein the reflective surface is curved.

6. The intravascular lithotripsy device of claim 1, wherein the reflective surface is configured to focus the sonic waves exterior to the housing distal to the distal end.

7. The intravascular lithotripsy device of claim 1 further comprising:
an irrigation lumen positioned at least partially within the housing and configured to conduct the fluid through the housing toward the distal end to flush a vicinity of a spark gap to inhibit gasses from accumulating in the vicinity of the spark gap and interfering with the sonic waves.

8. The intravascular lithotripsy device of claim 7, wherein the irrigation lumen is configured to continuously flush the fluid through the housing while the spark forms between the first exposed portion and the second exposed portion.

9. The intravascular lithotripsy device of claim 7, wherein the housing is configured to conduct the fluid away from the distal end after the fluid has passed from the irrigation lumen through the vicinity of the spark gap.

10. The intravascular lithotripsy device of claim 7 further comprising:
a drain lumen positioned at least partially within the housing, wherein the drain lumen is configured to receive the fluid conducted from the irrigation lumen to the distal end of the housing, wherein the drain lumen is configured to conduct the fluid from the distal end of the housing exterior to the housing.

11. The intravascular lithotripsy device of claim 1 further comprising:
a guidewire sheath configured to guide the intravascular lithotripsy device along a guidewire, wherein the guidewire sheath is offset from a longitudinal axis of the housing.

12. The intravascular lithotripsy device of claim 11, wherein the guidewire sheath is positioned outside of the housing.

13. An intravascular lithotripsy device configured to modify vascular plaque, comprising:
a housing configured to hold a fluid, the housing comprising a distal end;
a first insulated wire at least partially housed within the housing, wherein the first insulated wire comprises a first exposed portion positioned within the housing; and
a second insulated wire at least partially housed within the housing, wherein the first insulated wire comprises a second exposed portion positioned within the housing, wherein the first exposed portion and the second exposed portion are configured to form a spark adjacent to the distal end of the housing responsive to an energy differential between the first exposed portion and the second exposed portion exceeding a threshold, wherein the spark is configured to induce sonic waves configured to travel through the fluid,
wherein at least one of the first exposed portion or the second exposed portion faces away from the distal end of the housing.

14. The intravascular lithotripsy device of claim 13 further comprising a reflective surface positioned proximal to the first exposed portion and the second exposed portion, wherein the reflective surface is configured to reflect the sonic waves toward the distal end.

15. The intravascular lithotripsy device of claim 13, wherein the first exposed portion is at a proximally facing end of the first insulated wire, wherein the second exposed portion is at a proximally facing end of the second insulated wire, wherein the spark is configured to induce the sonic waves to travel proximally through the fluid away from the distal end.

16. The intravascular lithotripsy device of claim 13, wherein the first exposed portion faces the second exposed portion.

17. The intravascular lithotripsy device of claim 13, wherein the first exposed portion and the second exposed portion are configured to form the spark across a longitudinal axis of the housing.

18. The intravascular lithotripsy device of claim 13, wherein the first exposed portion and the second exposed portion are configured to form the spark independently from forming another spark.

19. The intravascular lithotripsy device of claim 13, wherein the first insulated wire is curved adjacent to the first exposed portion.

20. The intravascular lithotripsy device of claim 13 further comprising:
an irrigation lumen positioned at least partially within the housing and configured to conduct the fluid through the housing toward the distal end to flush a vicinity of a spark gap to inhibit gasses from accumulating in the vicinity of the spark gap and interfering with the sonic waves.

21. The intravascular lithotripsy device of claim 13 further comprising:
a guidewire sheath configured to guide the intravascular lithotripsy device along a guidewire, wherein the guidewire sheath is offset from a longitudinal axis of the housing.
